(19) United States
(12) Patent Application Publication
(10) Pub. No.: US 2003/0158218 A1
(43) Pub. Date: Aug. 21, 2003

(54) Thrombin inhibitors

(76) Inventors: Philippe G. Nantermet, Lansdale, PA (US); Harold G. Selnick, Ambler, PA (US); James C. Barrow, Harleysville, PA (US); Craig Coburn, Royersford, PA (US); Christopher Burgey, Philadelphia, PA (US); Kyle A. Robinson, Philadelphia, PA (US); Terry Alan Lyle, Lederach, PA (US)

Correspondence Address: MERCK AND CO INC, P O BOX 2000, RAHWAY, NJ, 070650907

(21) Appl. No.: 10/313,533

(22) Filed: Dec. 6, 2002

Related U.S. Application Data
(60) Provisional application No. 60/343,320, filed on Dec. 21, 2001.

(51) Int. Cl. A61K 31/506; A61K 31/4745; A61K 31/444; C07D 471/02; C 07D 4 1/02; C 07D 4 3/02

(52) U.S. Cl. 514/275; 514/300; 514/332; 514/352; 544/331; 546/122; 546/264; 546/312

(57) ABSTRACT

Compounds of the invention are useful in inhibiting thrombin and associated thrombotic occlusions having the following structure:

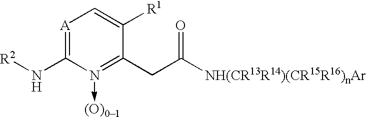

wherein R¹ is, for example, hydrogen, Cl, or cyano, and R² is, for example, hydrogen,

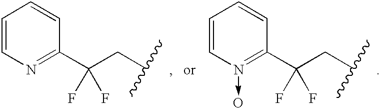

### BACKGROUND OF THE INVENTION

Thrombin is a serine protease present in blood plasma in the form of a precursor, prothrombin. Thrombin plays a central role in the mechanism of blood coagulation by converting the solution plasma protein, fibrinogen, into insoluble fibrin.

Edwards et al., J. Amer. Chem. Soc., (1992) vol. 114, pp. 1854-63, describes peptidyl a-ketobenzoxazoles which are reversible inhibitors of the serine proteases human leukocyte elastase and porcine pancreatic elastase. European Publication 363 284 describes analogs of peptidase substrates in which the nitrogen atom of the scissile amide group of the substrate peptide has been replaced by hydrogen or a substituted carbonyl moiety. Australian Publication 86245677 also describes peptidase inhibitors having an activated electrophilic ketone moiety such as fluoromethylene ketone or a-keto carboxyl derivatives. R. J. Brown et al., J. Med. Chem., Vol. 37, pages 1259-1261 (1994) describes orally active, non-peptidic inhibitors of human leukocyte elastase which contain trifluoromethylketone and pyridinone moieties. H. Mack et al., J. Enzyme Inhibition, Vol. 9, pages 73-86 (1995) describes rigid amidino-phenylalanine thrombin inhibitors which contain a pyridinone moiety as a central core structure.

### SUMMARY OF THE INVENTION

The invention includes compounds for inhibiting loss of blood platelets, inhibiting formation of blood platelet aggregates, inhibiting formation of fibrin, inhibiting thrombus formation, and inhibiting embolus formation in a mammal, comprising a compound of the invention in a pharmaceutically acceptable carrier. These compounds may optionally include anticoagulants, antiplatelet agents, and thrombolytic agents. The compounds can be added to blood, blood products, or mammalian organs in order to effect the desired inhibitions.

The invention also includes a compound for preventing or treating unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels, in a mammal, comprising a compound of the invention in a pharmaceutically acceptable carrier. These compounds may optionally include anticoagulants, antiplatelet agents, and thrombolytic agents.

The invention also includes a method for reducing the thrombogenicity of a surface in a mammal by attaching to the surface, either covalently or noncovalently, a compound of the invention.

### DETAILED DESCRIPTION OF THE INVENTION AND PREFERRED EMBODIMENTS

Compounds of the invention are useful as thrombin inhibitors and have therapeutic value in for example, preventing coronary artery disease. The invention includes compounds having the following structure:

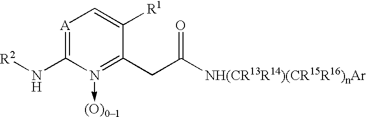

or a pharmaceutically acceptable salt thereof, wherein

R¹ is selected from the group consisting of hydrogen, halogen, cyano, C₁₋₄ alkyl, cyclo C₃₋₇ alkyl, and CF₃;

A is CH or N;

n is 0 or 1;

R² is hydrogen or —C(R⁷)(R⁸)C(R⁹)(R¹⁰)R¹¹, wherein

R⁷ and R⁸ are independently selected from the group consisting of hydrogen, halogen and C₁₋₄ alkyl unsubstituted or substituted with halogen, OR¹², N(R¹²)₂, COOR¹², CON(R¹²)₂, aryl or a heterocyclic ring, wherein R¹² is independently selected from the group consisting of hydrogen and C₁₋₄ alkyl,

R⁹ and R¹⁰ are independently selected from the group consisting of hydrogen, halogen, and C₁₋₄ alkyl, and

R¹¹ is aryl, C₁₋₄ alkyl, C₃₋₇ cycloalkyl, or heterocycle;

R¹³ and R¹⁴ are independently selected from the group consisting of hydrogen and C₁₋₂ alkyl unsubstituted or substituted with OR¹⁵ or COOR¹⁵, wherein R¹⁵ is hydrogen or C₁₋₄ alkyl;

R¹⁵ and R¹⁶ are independently selected from the group consisting of hydrogen, halogen and C₁₋₄ alkyl;

Ar is

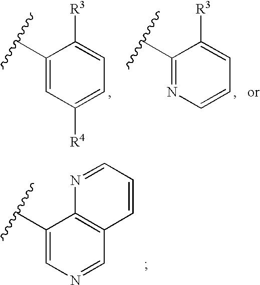

R³ is selected from the group consisting of

1) hydrogen,

2) halogen,

3) hydroxy,

4) C₁₋₄ alkyl,

5) C₁₋₄ alkoxy,

6) cyano,

7) —OCF₃,

8) —OCHF₂,

9) —OCH₂CF₃,

10) —C(R²³)(R²⁴)C(R²⁵)(R²⁶)N(R²⁷)(R²⁸) or —C(R²³)(R²⁴)N(R²⁷)(R²⁸) wherein

R²³ and R²⁴ are independently selected from the group consisting of

a) hydrogen,

b) F,

c) C₁₋₄ alkyl,

d) —CF₃,

e) —CHF₂,

f) C₃₋₇ cycloalkyl,

or R²³ and R²⁴ together form a 3-7 membered carbocyclic ring,

R²⁵ and R²⁶ are independently selected from the group consisting of

a) hydrogen,

b) C₁₋₄ alkyl

c) —CF₃,

d) —CHF₂,

e) —CH₂OH,

f) C₃₋₆ cycloalkyl,

or R²⁵ and R²⁶ together form a 3-7 membered carbocyclic ring,

R²⁷ and R²⁸ are independently selected from the group consisting of

a) hydrogen,

b) C₁₋₆ alkyl, unsubstituted or substituted with —OH, C₃₋₇ cycloalkyl, or —C(O)OR³, wherein R³¹ is selected from the group consisting of hydrogen and C₁₋₆ alkyl, and

c) C₃₋₇ cycloalkyl,

or R²⁷ and R²⁸ are joined to form a 4-7 membered heterocyclic ring which is unsubstituted or substituted with hydroxyl or halogen,

11)—SR¹⁷,

12) —SOR¹⁷,

13) —SO₂R¹⁷,

14) —OR¹⁸,

15) —SR¹⁸,

16) —NHR¹⁸,

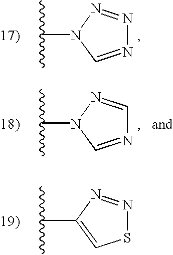

wherein

R¹⁷ is C₁₋₄ alkyl unsubstituted or substituted with —C(CH₃)₂NH₂, —C(CH₃)₂OH, —C(CH₃)₂NHCOCF₃, or CF₃, and

R¹⁸ is phenyl unsubstituted or substituted with one or more of C₁₋₄ alkyl, C₁₋₄ alkoxy, halogen, hydroxy, COOH, CONH₂, CH₂OH or COOR¹⁹, wherein R¹⁹ is C₁₋₄ alkyl;

R⁴ is selected from the group consisting of

1) hydrogen,

2) halogen,

3) hydroxy,

4) C₁₋₄ alkyl,

5) C₁₋₄ alkoxy,

6) cyano,

7) —OCF₃,

8) —OCHF₂,

9) —OCH₂CF₃.

In a class of compounds of the invention, R¹ is hydrogen, halogen or cyano, and n is 0.

In a subclass of this class, R⁷, R⁸, R⁹, and R¹⁰ are independently selected from the group consisting of hydrogen and halogen, and

R¹¹ is

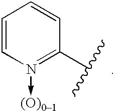

In a group of this subclass, R¹³ and R¹⁴ are hydrogen.

In a subgroup of this group, R³ is selected from the group consisting of hydrogen, —CH₂NH₂, —CH₂CH₂NH₂, —CH₂ NHCH₃, halogen,

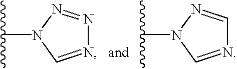

In a family of this subgroup, R⁴ is hydrogen or halogen.

In a subfamily of this family, R¹ is hydrogen, Cl, or cyano; and

R² is hydrogen,

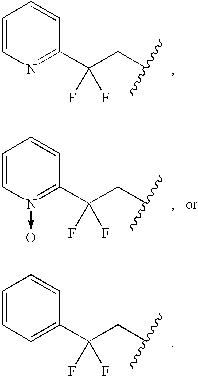

In a collection of this subfamily,

Ar is

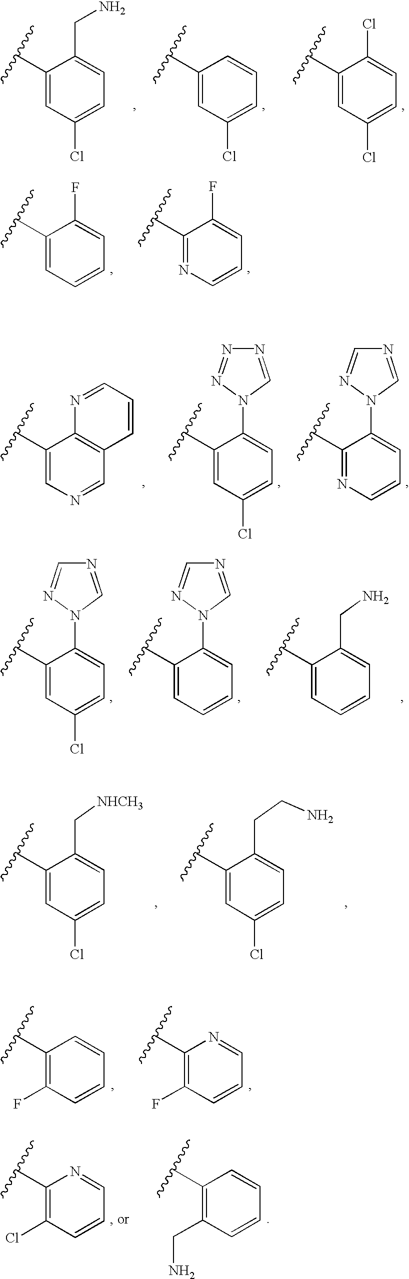

Specific examples of compounds of the invention include

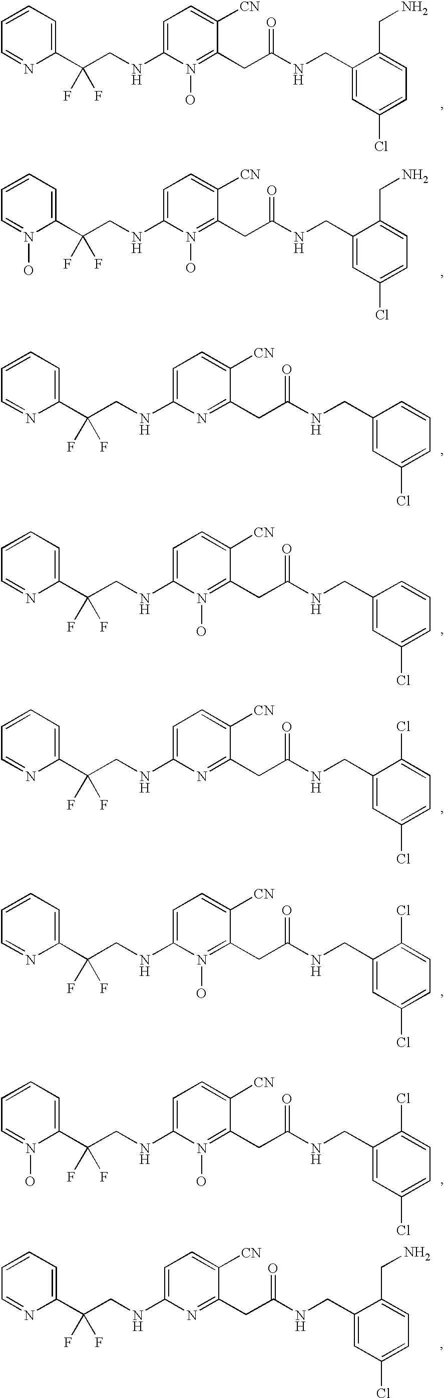

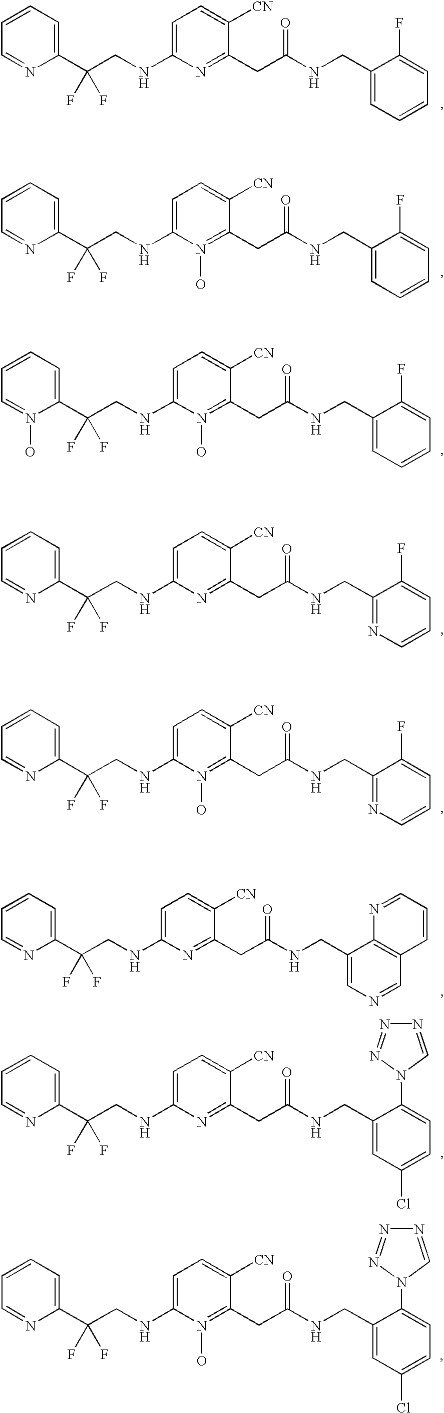

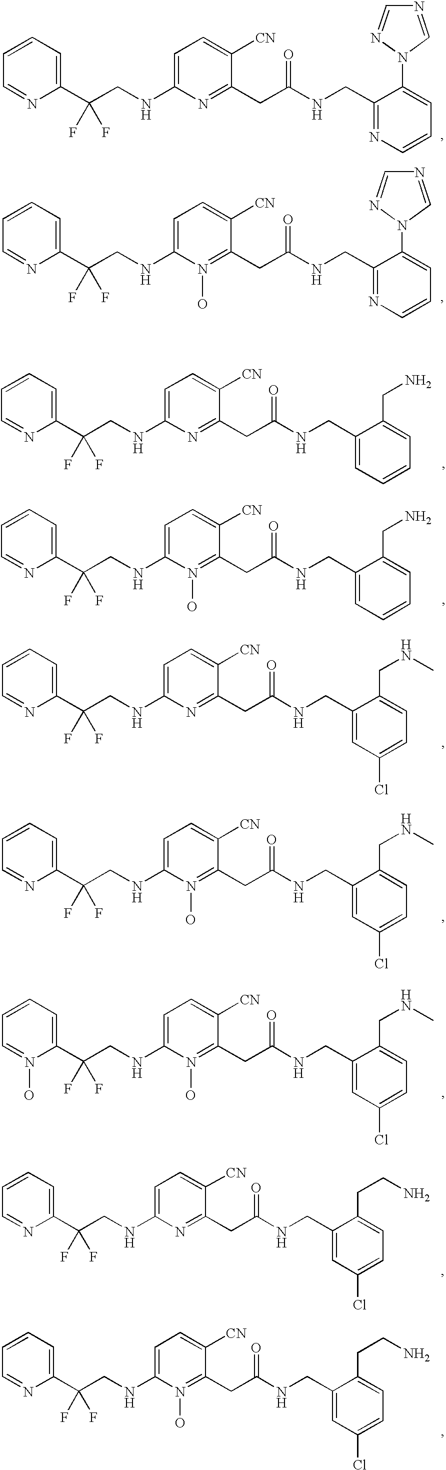

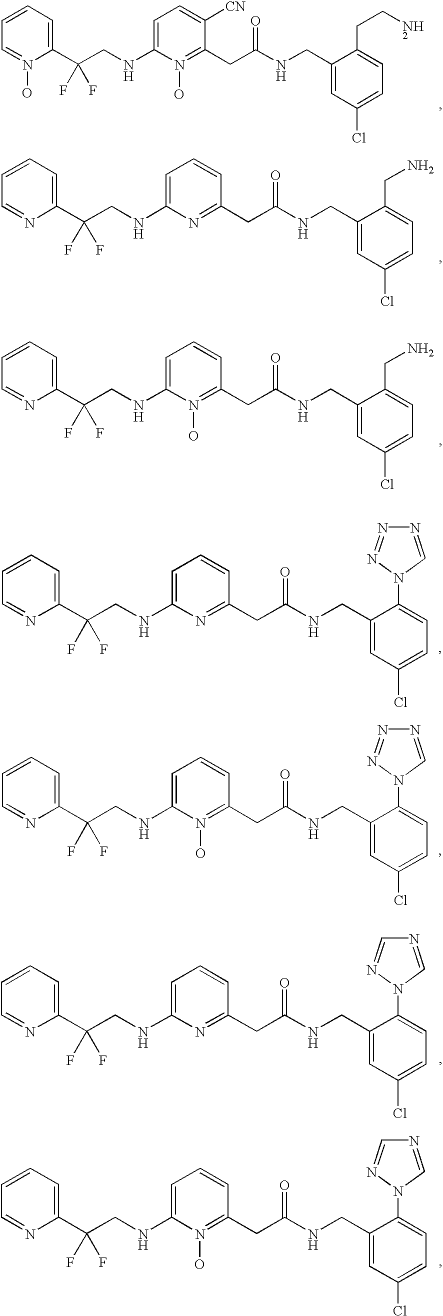

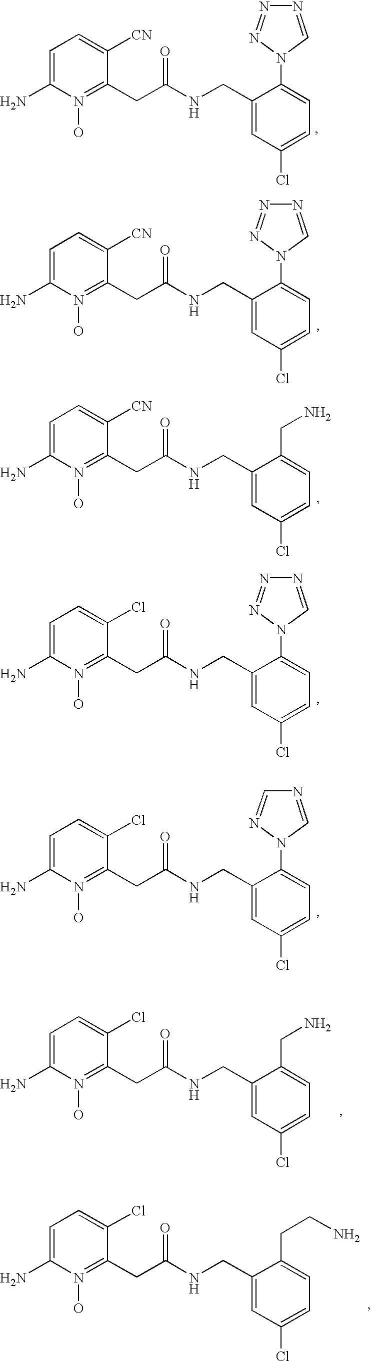

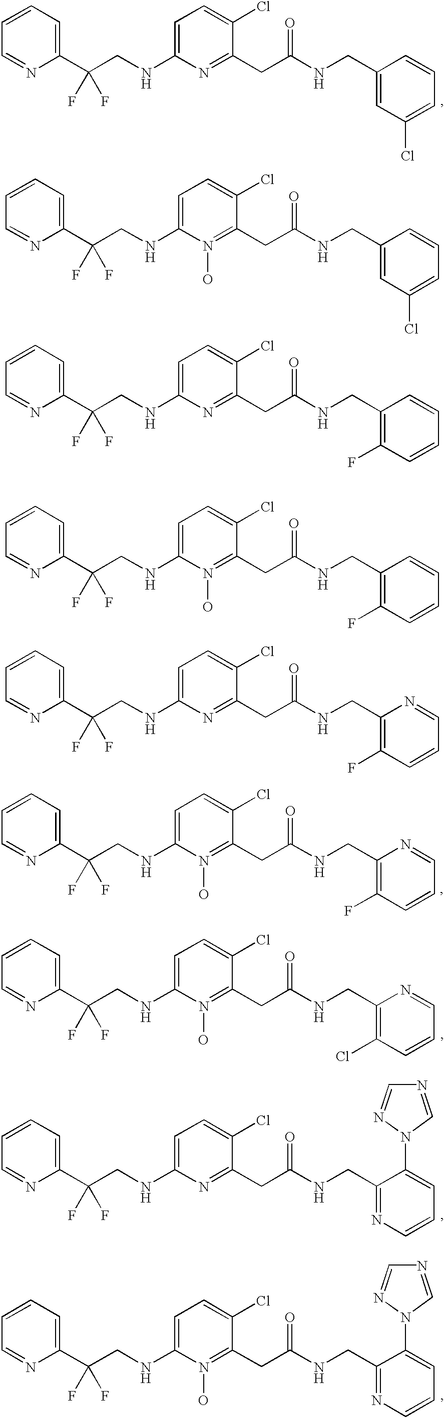

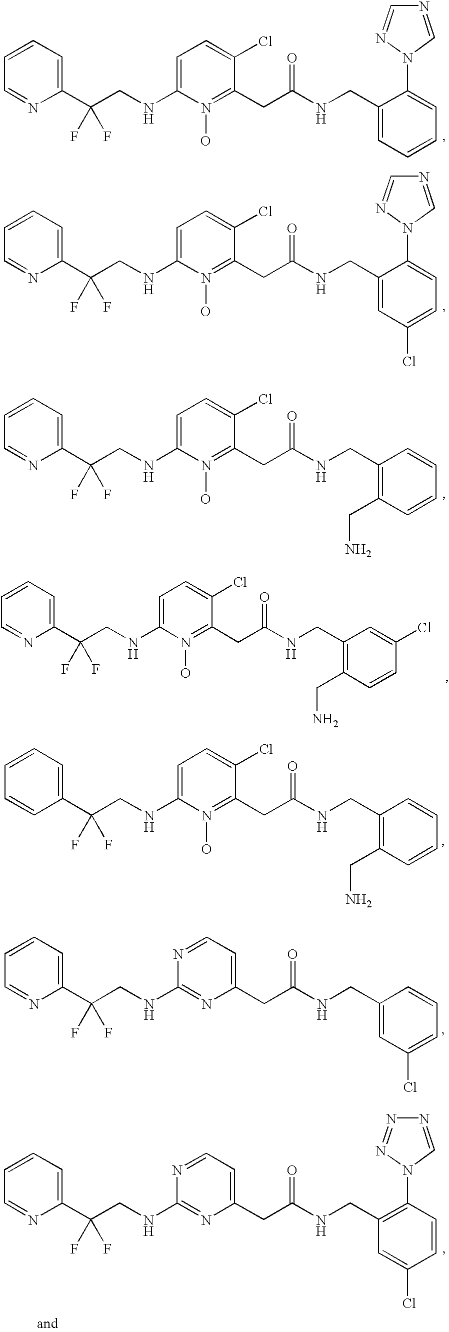

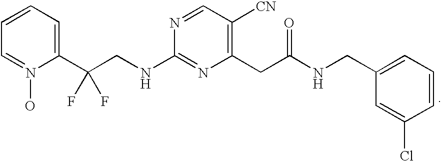

The compounds of the present invention, may have chiral centers and occur as racemates, racemic mixtures and as individual diastereomers, or enantiomers with all isomeric forms being included in the present invention. The compounds of the present invention may also have polymorphic crystalline forms, with all polymorphic crystalline forms being included in the present invention.

When any variable occurs more than one time in any constituent or in formula I, its definition on each occurrence is independent of its definition at every other occurrence. Also, combinations of substituents and/or variables are permissible only if such combinations result in stable compounds.

Some abbreviations that may appear in this application are as follows:

### Abbreviations

Designation

| | |
|---|---|
| (Boc)₂O | di-t-butyl dicarbonate |
| 3-ClPhCH₂NH₂ | m-chlorobenzylamine |
| CO | carbon monoxide |
| CoCl₂ | cobalt (II) chloride |
| Cs₂CO₃ | cesium carbonate |
| DBU | 1,8-diazabicyclo[5.4.0]undec-7-ene |
| DIBAL | diisobutyl aluminum hydride |
| DMAP | dimethylaminopyridine |
| DMF | dimethylformamide |
| DPPA | diphenylphosphoryl azide |
| EDC | 1-ethyl-3-(3-dimethylaminopropyl)carbodiimide hydro- |
| | chloride |
| (EtO)₂CO | diethyl carbonate |
| H₂ | hydrogen |
| HCl | hydrochloric acid |
| HOAt | 1-hydroxy-7-azabenzotriazole |
| H₂SO₄ | sulfuric acid |
| LDA | lithium diisopropyl amide |
| LiBH₄ | lithium borohydride |
| LiCl | lithium chloride |
| LiOH | lithium hydroxide |
| mCPBA | m-chloroperoxybenzoic acid |
| MeOH | methanol |
| n-BuLi | n-butyllithium |
| NaBH₄ | sodium borohydride |
| NaH | sodium hydride |
| NaHMDS | sodium hexamethyl disylazide |
| NaN₃ | sodium azide |
| Na₂SO₃ | sodium sulfite |
| Na₂SO₄ | sodium sulfate |
| NCS | N-chlorosuccinimide |
| NH₄Cl | ammonium chloride |
| POCl₃ | phosphorous oxychloride |
| P(Ph)₃ | triphenyl phosphine |
| Pd/C | palladium on activated carbon catalyst |
| Ra Ni | Raney nickel |
| TFA | trifluoroacetic acid |
| THF | tetrahydrofuran |
| Zn(CN)₂ | zinc cyanide |

As used herein except where noted, “alkyl” is intended to include both branched- and straight-chain saturated aliphatic hydrocarbon groups having the specified number of carbon atoms (Me is methyl, Et is ethyl, Pr is propyl, Bu is butyl), unsubstituted or substituted with C₁₋₄ alkyl or halogen; “alkoxy” represents a linear or branched alkyl group of indicated number of carbon atoms attached through an oxygen bridge; “halogen”, as used herein, means fluoro, chloro, bromo and iodo; and “counterion” is used to represent a small, single negatively-charged species, such as chloride, bromide, hydroxide, acetate, trifluoroacetate, perchlorate, nitrate, benzoate, maleate, sulfate, tartrate, hemitartrate, benzene sulfonate, and the like.

The term “cycloC₃₋₇alkyl” is intended to include cyclopropyl, cyclobutyl, cyclopentyl, cyclohexyl and cycloheptyl, and the like, unsubstituted or substituted with C₁₋₄ alkyl or halogen.

The term “aryl” as used herein except where noted, represents a stable 6- to 10-membered mono- or bicyclic ring system such as phenyl, or naphthyl. The aryl ring can be unsubstituted or substituted with one or more of C₁₋₄ lower alkyl; hydroxy; alkoxy; halogen; amino.

The term “heterocycle” or “heterocyclic ring”, as used herein except where noted, represents a stable 5- to 7-membered mono- or bicyclic or stable 7- to 10-membered bicyclic heterocyclic ring system unsubstituted or substituted with C₁₋₄ alkyl or halogen, any ring of which may be saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Especially useful are rings containing one oxygen or sulfur, one to four nitrogen atoms, or one oxygen or sulfur combined with one or two nitrogen atoms. The heterocyclic ring may be attached at any heteroatom or carbon atom which results in the creation of a stable structure. Examples of such heterocyclic groups include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyridyl N-oxide, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, tetrazole, thienyl, benzothienyl, thiamorpholinyl, thiamorpholinyl sulfoxide, thiamorpholinyl sulfone, and oxadiazolyl. Morpholino is the same as morpholinyl.

In this specification methyl substituents may be represented by

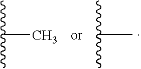

For example, the structures

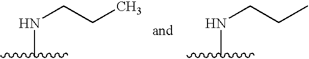

have equivalent meanings.

The pyridyl N-oxide portion of the compounds of the invention are structurally depicted using conventional representations

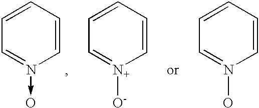

which have equivalent meanings.

The pharmaceutically-acceptable salts of the compounds of Formula I (in the form of water- or oil-soluble or dispersible products) include the conventional non-toxic salts such as those derived from inorganic acids, e.g. hydrochloric, hydrobromoic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Thrombin Inhibitors—Therapeutic Uses—Method of Using

Anticoagulant therapy is indicated for the treatment and prevention of a variety of thrombotic conditions, particularly coronary artery and cerebrovascular disease. Those experienced in this field are readily aware of the circumstances requiring anticoagulant therapy. The term “patient” used herein is taken to mean mammals such as primates, including humans, sheep, horses, cattle, pigs, dogs, cats, rats, and mice.

Thrombin inhibition is useful not only in the anticoagulant therapy of individuals having thrombotic conditions, but is useful whenever inhibition of blood coagulation is required such as to prevent coagulation of stored whole blood and to prevent coagulation in other biological samples for testing or storage. Thus, the thrombin inhibitors can be added to or contacted with any medium containing or suspected of containing thrombin and in which it is desired that blood coagulation be inhibited, e.g., when contacting the mammal's blood with material selected from the group consisting of vascular grafts, stents, orthopedic prosthesis, cardiac prosthesis, and extracorporeal circulation systems.

Compounds of the invention are useful for treating or preventing venous thromboembolism (e.g. obstruction or occlusion of a vein by a detached thrombus; obstruction or occlusion of a lung artery by a detached thrombus), cardiogenic thromboembolism (e.g. obstruction or occlusion of the heart by a detached thrombus), arterial thrombosis (e.g. formation of a thrombus within an artery that may cause infarction of tissue supplied by the artery), atherosclerosis (e.g. arteriosclerosis characterized by irregularly distributed lipid deposits) in mammals, and for lowering the propensity of devices that come into contact with blood to clot blood.

Examples of venous thromboembolism which may be treated or prevented with compounds of the invention include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, compounds of the invention are useful for maintaining patency of indwelling catheters.

Examples of cardiogenic thromboembolism which may be treated or prevented with compounds of the invention include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

Examples of arterial thrombosis include unstable angina (severe constrictive pain in chest of coronary origin), myocardial infarction (heart muscle cell death resulting from insufficient blood supply), ischemic heart disease (local anemia due to obstruction (such as by arterial narrowing) of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterial thrombosis, compounds of the invention are useful for maintaining patency in arterioyenous cannulas.

Examples of atherosclerosis include arteriosclerosis.

Examples of devices that come into contact with blood include vascular grafts, stents, orthopedic prosthesis, cardiac prosthesis, and extracorporeal circulation systems

The thrombin inhibitors of the invention can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, or intramuscular form, all using forms well known to those of ordinary skill in the pharmaceutical arts. An effective but non-toxic amount of the compound desired can be employed as an anti-aggregation agent. For treating ocular build up of fibrin, the compounds may be administered intraocularly or topically as well as orally or parenterally.

The thrombin inhibitors can be administered in the form of a depot injection or implant preparation which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The thrombin inhibitors can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The thrombin inhibitors may also be delivered by the use of monoclonal antibodies as individual carriers to which the compound molecules are coupled. The thrombin inhibitors may also be coupled with soluble polymers as targetable drug carriers. Such polymers can include polyvinlypyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the thrombin inhibitors may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The dosage regimen utilizing the thrombin inhibitors is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the thrombin inhibitors, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-0.5 mg/kg/day (unless specificed otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/kg/day. A suitably prepared medicament for once a day administration would thus contain between 0.8 mg and 2.4 g, preferably between 2 mg and 600 mg, more preferably between 8 mg and 200 mg, and most preferably 8 mg and 40 mg, e.g., 8 mg, 10 mg, 20 mg and 40 mg. Advantageously, the thrombin inhibitors may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg.

Intravenously, the patient would receive the active ingredient in quantities sufficient to deliver between 0.025-7.5 mg/kg/day, preferably 0.1-2.5 mg/kg/day, and more preferably 0.1-0.5 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/ml, e.g. 0.1 mg/ml, 0.3 mg/ml, and 0.6 mg/ml, and administered in amounts per day of between 0.01 ml/kg patient weight and 10.0 ml/kg patient weight, e.g. 0.1 ml/kg, 0.2 ml/kg, 0.5 ml/kg. In one example, an 80 kg patient, receiving 8 ml twice a day of an intravenous formulation having a concentration of active ingredient of 0.5 mg/ml, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. Consideration should be given to the solubility of the drug in choosing an The choice of appropriate buffer and pH of a formulation, depending on solubility of the drug to be administered, is readily made by a person having ordinary skill in the art.

The compounds can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The thrombin inhibitors are typically administered as active ingredients in admixture with suitable pharmaceutical diluents, excipients or carriers (collectively referred to herein as “carrier” materials) suitably selected with respect to the intended form of administration, that is, oral tablets, capsules, elixers, syrups and the like, and consistent with convention pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, distintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch methyl cellulose, agar, bentonite, xanthan gum and the like.

The compounds claimed in this invention can be prepared according to the following general procedures.

General Preparations

Protection of 2-methyl-3-cyano-6-amino-pyridine (Tetrahedron Lett., 1999, 40(47), 8193-8195) as its Boc derivative (Scheme 1) using standard procedure, followed by deprotonation at the methyl position and quench with diethylcarbonate provides 2-(6-tert-Butoxycarbonylamino-3-cyano-pyridin-2-yl)-acetic acid ethyl ester (2). Deprotonation at the NHBoc position with NaH and treatment with an alkyl triflate, followed by hydrolysis with 1N LiOH in THF gives access to carboxylic acid of type 3. Standard EDC/HOAt coupling of a benzyl amine or any amine, followed by Boc removal with anhydrous TFA leads to aminopyridine derivatives of type 4. Oxidation with metachloroperbenzoic acid occurs first on the central pyridyl ring affording N-oxide derivatives of type 5. Continued exposure to more metachloroperbenzoic acid in the same pot or in a second step gives rise to bis N-oxide derivatives of type 6. For derivatives containing a P1 group presenting a free amino group such as 7, selective Boc protection is accomplished before mCPBA oxidation.

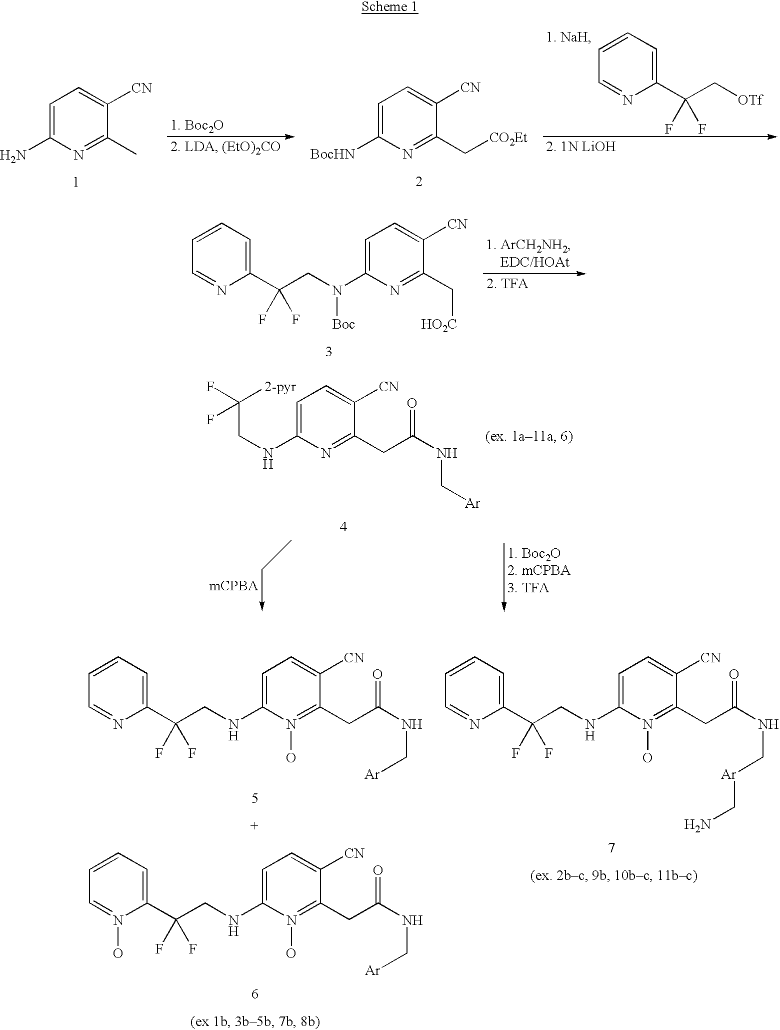

Alternatively, hydrolysis of ester of type 2 with 1N LiOH in THF, followed by EDC/HOAt coupling of a benzyl amine or any amine, followed by pyridyl N-oxidation with metachloroperbenzoic acid, and finally Boc removal with anhydrous TFA provides N-oxide derivatives of type 8.

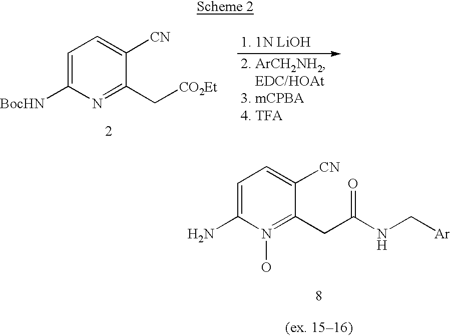

Using an identical synthetic scheme but starting with 2-methyl-6-amino-pyridine (2, Scheme 3) gives access to pyridyl derivatives of type 11 and N-oxide derivatives of type 12 and 13. N-oxide derivatives of type 14 can also be obtained according to Scheme 3.

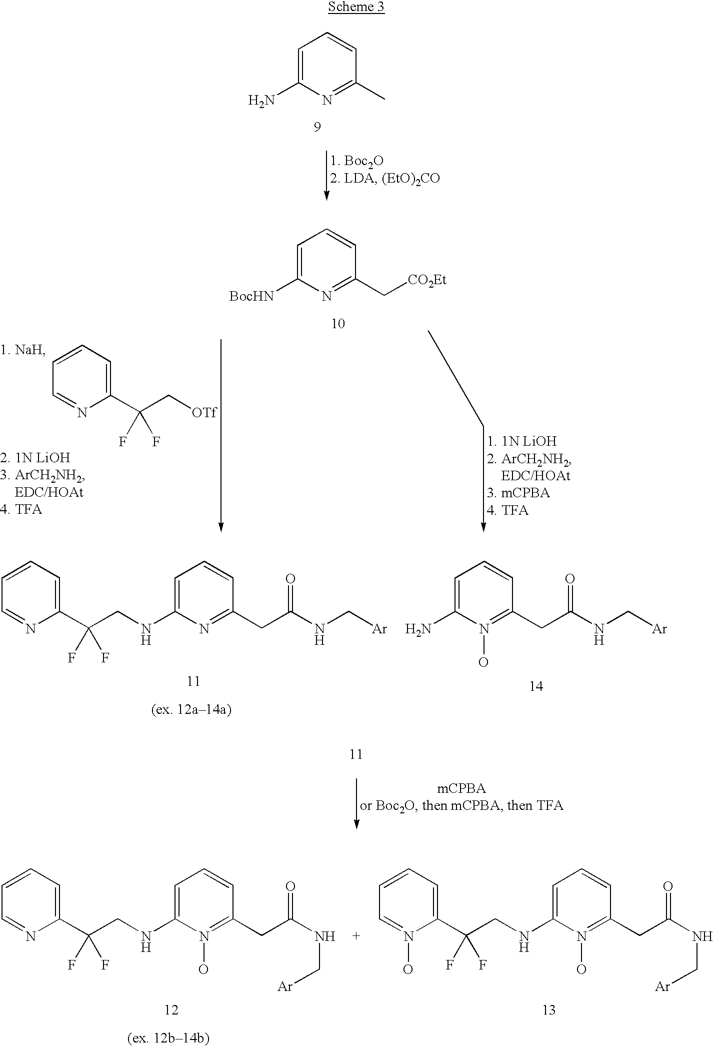

A similar set of derivatives can be obtained from 2-methyl-3-chloro-6-amino-pyridine (prepared by chlorination of 2-methyl-6-amino-pyridine at the 3-position with N-chlorosuccinimide), as shown in Scheme 4.

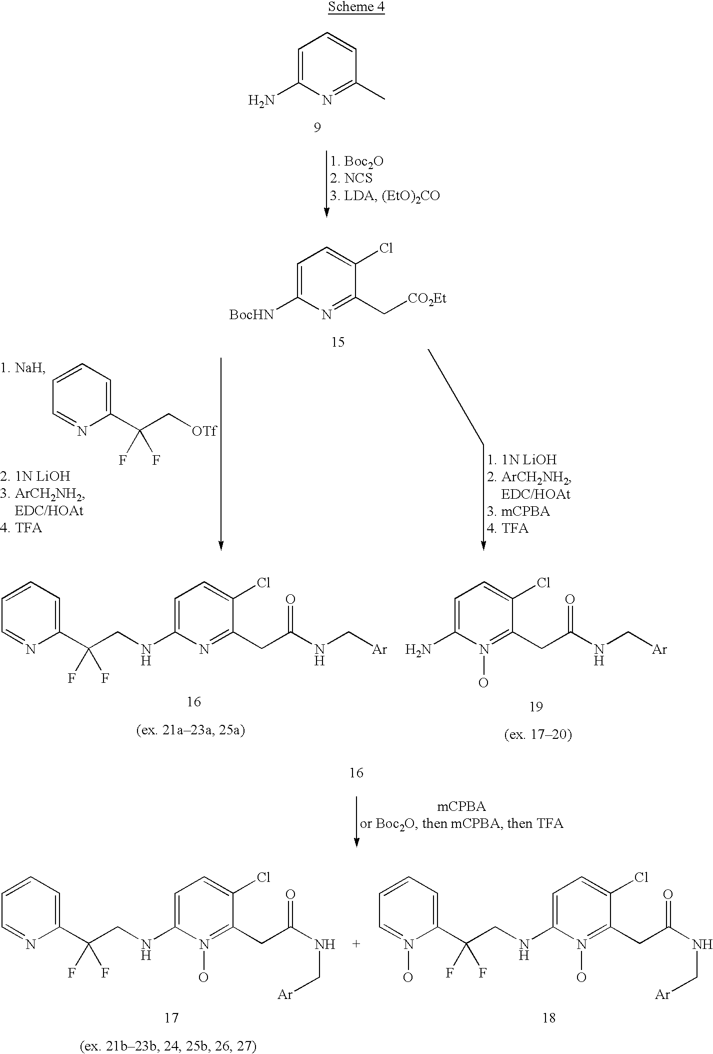

Pyrimidines of type 23 can be prepared according to scheme 5. Deprotonation of thiomethyl-pyrimidine 20 at the methyl position followed by quenching with diethyl carbonate and subsequent oxidation of the thiomethyl moiety to the corresponding sulfone provides ester 21. Sulfone discplacement with a P3 amine such as 22 provides aminopyrimidine 23. Hydrolysis and P1 group installation as described before leads to pyrimidine derivatives of type 24.

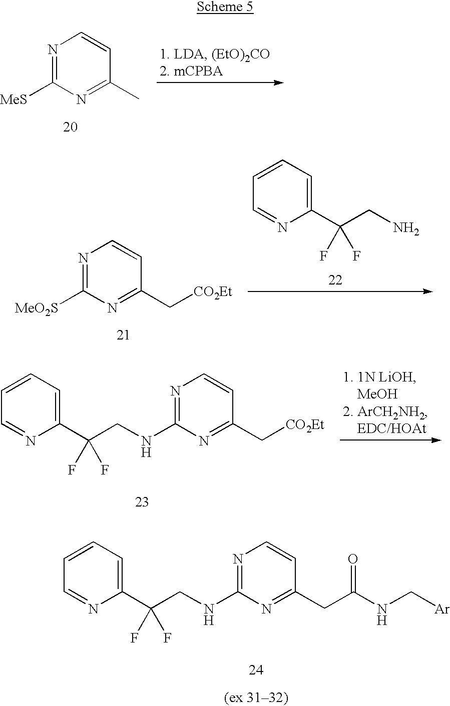

Cyanopyrimidines of type 29 can be prepared as shown in scheme 6. Condensation of 2-methyl-2-thiopseudourea sulfate and ethyl(ethoxy-methylene)-cyanoacetate followed by chlorination of the resulting hydroxypyrimidine provides chloroypyrimidine 25. The ethyl ester moiety is installed via the ethyl/TMS malonate to provide 26. Oxidation of the thiomethyl moiety followed by displacement with amine 27 leads to ester 28 which can be hydrolyzed and coupled to various P1 benzyl amine to afford cyanopyrimidines of type 29.

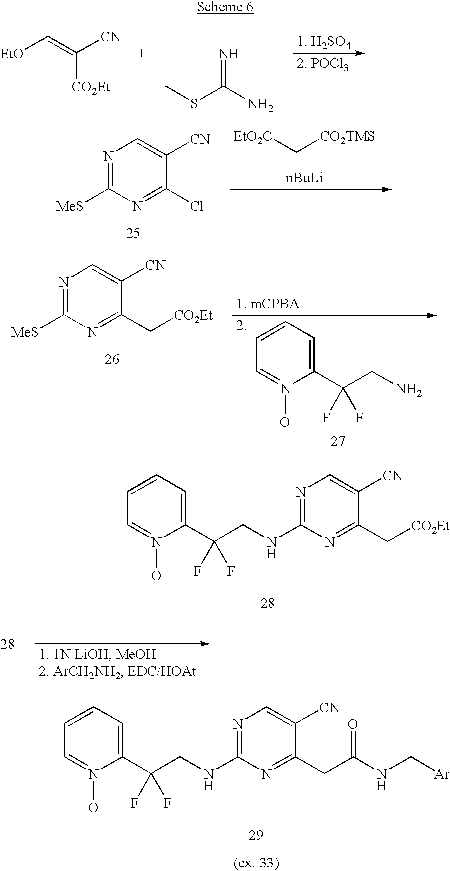

Preparation of Intermediates

The following scheme shows the preparation of various intermediates:

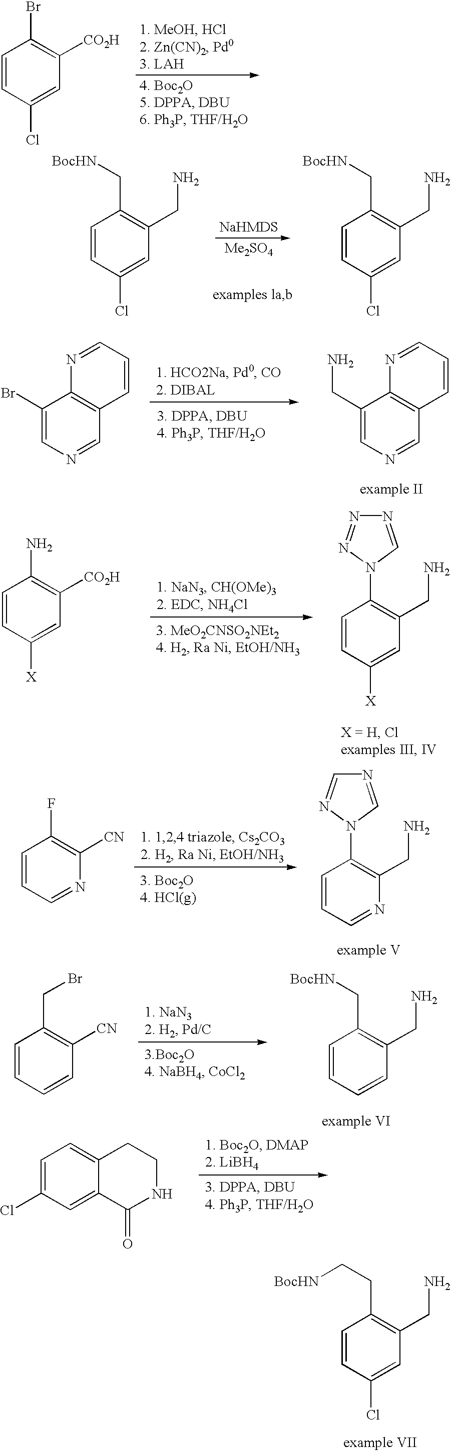

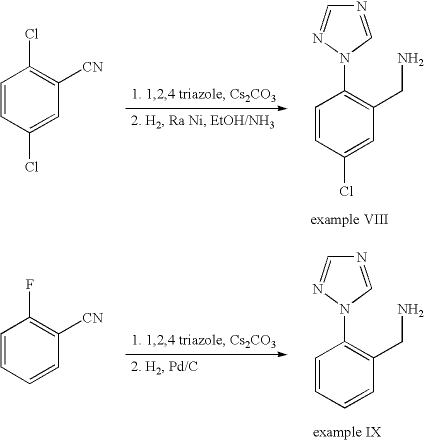

Unless otherwise stated, all NMR determinations were made using 400 MHz field strength.

### EXAMPLE Ia

tert-Butyl 2-(aminomethyl)-4-chlorobenzylcarbamate

Step A: Preparation of 2-bromo-5-chlorobenzoate

Through a solution of 2-bromo-5-chlorobenzoic acid (11 g, 46.7 mmol) in methanol (250 ml) was bubbled HCl gas. The reaction was allowed to warm to room temperature and stirred overnight. The reaction mixture is concentrated in vacuo to give an orange oil, which is purified by flash chromatography (silica gel, hexane) to give the title compound as a colorless oil.

¹H NMR (CDCl₃, 400 MHz): δ 7.78 (d, 1H, J=2.6 Hz); 7.59 (d, 1H, J=12.81 Hz);7.30 (dd, 1H, J=8.6, 2.5 Hz); 3.94 (s, 3H)

Step B: Preparation of Methyl 5-chloro-2-cyanobenzoate

To a solution of methyl 2-bromo-5-chlorobenzoate (1.15 g, 4.6 mmol) in degassed DMF was added zinc cyanide (282 mg, 2.40 mmol) and palladium tetrakis triphenylphosphine (100 mg, 0.086 mmol) and the reaction is stirred at 90° C. over night. The reaction was partitioned between ethyl acetate and water. The organic was concentrated in vacuo and purified by flash chromatography eluting a gradient to 10 to 25% ethyl acetate in hexane yielding a white solid (methyl 5-chloro-2-cyanobenzoate.

¹H NMR (CDCl₃, 400 MHz): δ 8.13 (d, 1H, J=1.83 Hz); 3.09 (d, 1H, J=8.24 Hz); 7.29 (dd, 1H, J=8.34, 2.10 Hz); 4.02 (s, 3H)

Step C: Preparation of 2-(aminomethyl)-5-chlorophenyl]methanol

To LAH (1 M/Et₂O, 104.4 ml, 104.4 mmol) in anhydrous THF (300 ml) at 0C was added methyl 5-chloro-2-cyanobenzoate (9.28 g, 0.512 mmol) maintaining the temperature below 20° C. After one half hour, quenched at 0° C. with water (3.97 ml), NaOH (1N, 11.9 ml, 11.9 mmol) and water (3.97 ml). A precipitate was filtered out and washed with THF. The filtrate was concentrated in vacuo and was used immediately in the next step.

¹H NMR (CDCl₃, 400 MHz): δ 7.17-7.36 (m, 3H); 4.60 (s, 2H); 3.98 (s, 2H);

Step D: Preparation of tert-butyl 4-chloro-2-(hydroxymethyl)benzylcarbamate

To a solution of [2-(aminomethyl)-5-chlorophenyl]methanol in dichloromethane (200 ml), was added di-tert-butyl-dicarbonate (11.38 g, 52.18 mmol) at room temperature. After one hour, the reaction was partitioned. The organic layer was concentrated in vacuo and purified by flash chromatography eluting a gradient of ethyl acetate/hexane which gave a brown oil, which was taken up in dichloromethane (500 ml) and treated with activated charcoal yielding a pink solid.

¹H NMR (CDCl₃, 400 MHz): δ 7.36 (s, 1H); 7.2-7.5 (m, 2H); 4.69 (b s, 2H); 4.32 (d, 2H, J=6.04 Hz); 1.43 (s, 9H).

Step E: Preparation of tert-Butyl 2-(azidomethyl)-4-chlorobenzylcarbamate

To a solution of tert-butyl 4-chloro-2-(hydroxymethyl)benzylcarbamate (10 g, 36.8 mmol) in anhydrous THF (100 ml) was added DPPA (8.3 ml, 38.6 mmol) and DBU (5.79 ml, 38.6 mmol). The mixture was stirred overnight and then was partitioned between ethyl acetate and water. The organic layer was washed with brine, and was concentrated in vacuo to a crude oil (14.6 g). Purification was accomplished by silica gel chromatography, eluting a gradient of ethyl acetate-hexane (10, 15, 20, 25, 50%) to give tert-butyl 2-(aminomethyl)-4-chlorobenzylcarbamate.

¹H NMR (CDCl₃, 400 MHz): δ 7.25-7.39 (m, 3H); 4.41 (s, 2H), 4.32 (d, 2H, J=5.86 Hz); 1.45 (s, 9H).

Step F: Preparation of tert-Butyl 2-(aminomethyl)-4-chlorobenzylcarbamate

To a solution of tert-butyl 2-(azidomethyl)-4-chlorobenzylcarbamate (10.9 g, 36.73 mmol) in THF (60 ml) and water (6 ml) was added triphenylphospine (10.59 g, 40.40 mmol). The reaction was heated to 65° C. and stirred overnight at room temperature. The reaction was concentrated in vacuo and flashed with 4% (10%NH₄OH/MeOH)/dichlor-omethane. A second purification using silica gel column chromatography with a careful gradient of 3 to 5% (10%NH₄OH/MeOH)/dichloro methane gave the title compound.

¹H NMR (CDCl₃, 400 MHz) δ 7.21-7.52 (m, 3H); 4.32 (b d, 2H); 3.90 (s, 2H); 1.44 (s, 9H).

### EXAMPLE Ib

tert-Butyl 2-(methylaminomethyl)-4-chlorobenzylcarbamate

To a solution of tert-Butyl 2-(aminomethyl)-4-chlorobenzylcarbamate (762 mg, 2.81 mmol) in DMF (8 ml) cooled to 0° C. was added sodium bis(trimethylsilyl)amide (2.81 ml, 2.81 mmol, 1M in THF). After stirring at 0° C. for 15 min, dimethylsulfate (266 ul, 2.81 mmol) was added dropwise. The reaction mixture was stirred at 0° C. for 30 ml, diluted with water and extracted with chloroform three times. The organic layer was washed with aqueous LiCl, dried on Na₂SO₄, concentrated in vacuo and purified by flash chromatography (silica gel, 1% MeOH (containing 10% NH₄OH) in dichloromethane to 4%) to give tert-Butyl 2-(methylaminomethyl)-4-chlorobenzylcarbamate. ¹H NMR (CDCl₃, 400 MHz): δ 7.40 (d, J=2.5 Hz, 1H); 7.22 (dd, J=8.4, 2.5 Hz, 1H); 7.08 (d, J=8.4 Hz, 1H); 4.50 (s, 2H); 3.85 (s, 2H); 2.80 (s, 3H); 1.48 (s, 9H).

### EXAMPLE II

8-aminomethyl-1,6-napthyridine

Step A: Preparation of 8-hydroxymethyl-1,6-napthyridine:

Through a solution of 3 g (14 mmol) 8-bromo-1,6-napthyridine in 700 mL DMF was passed a steady stream of CO gas for 1 h. To this was added 1.8 g (26 mmol) sodium formate and 1.5 g (2.1 mmol) (Ph₃P)₂PdCl₂. The resulting mixture was heated to 95° C. while continuing to bubble CO gas through the mixture for 4 h., then concentrated in vacuo. The residue was treated with 100 mL CH₂Cl₂ and filtered through celite (2×100 mL CH₂Cl₂ wash). The resulting filtrates were combined and concentrated to give 3.8 g orange oil that was taken up in 100 mL dry CH₂Cl₂ and cooled to −78 □C whereupon 14 mL (14 mmol, 1M solution in CH₂Cl₂) diisobutylalumnium hydride was quickly added by syringe. The resulting mixture was stirred at −78° C. for 30 min., then poured into a well stirred mixture of 600 mL saturated aqueous sodium/potassium tartrate and 600 mL EtOAc, stirred at room temperature for 6 hours, then filtered through Celite. The layers were then separated and the aqueous layer extracted 3×400 mL EtOAc. The combined EtOAc extracts were dried over Na₂SO₄, filtered and concentrated in vacuo. Purification by flash chromatography (50×120 mm silica gel, linear gradient 3-8% MeOH:CH₂Cl₂) yielded 8-hydroxymethyl-1,6-napthyridine. ¹H NMR (CDCl₃, 400 MHz) δ 9.25 (s, 1H); 9.09 (dd, 1H, J=4.3 and 1.74 Hz); 8.68 (s, 1H); 8.35 (dd, 1H, J=8.3 and 1.74 Hz); 7.60 (dd, 1H, J=8.3 and 4.3 Hz); 5.22 (d, 2H, J=6.59 Hz); 4.42 (t, 10H, J=6.58 Hz). MS ES+M+1=160.9.

Step B: Preparation of 8-azidomethyl-1,6-napthyridine

To a solution of 0.93 g (5.8 mmol) 8-hydroxymethyl-1,6-napthyridine in 20 mL THF was added 1.5 mL (7 mmol) DPPA and 1.2 mL (6.7 mmol) DBU. The reaction mixture was allowed to stir at room temperature 18 hours, then another 0.3 mL DPPA and 0.25 mL DBU were added and the reaction mixture heated to 50° C. for 8 hours then cooled to room temperature then another 0.3 mL DPPA and 0.25 mL DBU were added and the reaction mixture was allowed to stir 18 more hours at room temperature. The resulting solution was then diluted with 200 mL EtOAc, washed with saturated NaHCO₃ solution, and brine, dried over Na₂SO₄, filtered and concentrated in vacuo. Purification by flash chromatography (40×120 mm silica gel, linear gradient 2-15% MeOH:CH₂Cl₂) yielded 8-hydroxymethyl-1,6-napthyridine and 8-azidomethyl-1,6-napthyridine. ¹H NMR (CDCl₃, 400 MHz) δ 9.30 (s, 1H); 9.16 (dd, 1H, J=4.3 and 1.8 Hz); 8.78 (s, 1H); 8.35 (dd, 1H, J=8.3 and 1.74 Hz); 7.61 (dd, 1H, J=8.3 and 4.3 Hz); 5.00 (s, 2H).

Step C: Preparation of 8-aminomethyl-1,6-napthyridine

To a solution of 1.1 g (5.9 mmol) 8-azidomethyl-1,6-napthyridine in 20 mL THF was added 2 mL H₂O and 3 g PPh₃. The resulting solution was alloed to stir overnight at room temperature, then concentrated in vacuo. Purification by flash chromatography (50×140 mm silica gel, linear gradient 5-20% (10% NH₄OH in MeOH):CH₂Cl₂) yielded 8-aminomethyl-1,6-napthyridine. ¹H NMR (CDCl₃, 400 MHz) δ 9.22 (s, 1H); 9.13 (dd, 1H, J=4.3 and 1.8 Hz); 8.70 (s, 1H); 8.32 (dd, 1H, J=8.24 and 1.83 Hz); 7.61 (dd, 1H, J=8.24 and 4.3 Hz); 4.40 (s, 2H).

### EXAMPLE III

2-Tetrazol-1-yl-benzylamine

Step A: Preparation of 2-Tetrazol-1-yl-benzoic Acid

A suspension of 2-aminobenzoic acid (6.0 g, 0.044 mol), trimethyl orthoformate (14.2 ml, 0.13 mol) and sodium azide (8.4 g, 0.13 mol) in glacial acetic acid (150 ml) was stirred at room temperature for 2 h. Filtration and concentration from toluene gave 2-tetrazol-1-yl-benzoic acid; ¹H NMR (CD₃OD, 400 MHz) δ 9.47 (s, 1H), 8.19 (dd, 1H, J=7.7 Hz, J=1.6 Hz), 7.79 (m, 2H), 7.61 (dd, 1H, J=7.7 Hz, J=1.5 Hz).

Step B: Preparation of 2-Tetrazol-1-yl-benzamide

A solution of 2-tetrazol-1-yl-benzoic acid (1.0 g, 5.2 mmol), ammonium chloride (0.56 g, 10.4 mmol), 1-(3-dimethylaminopropyl)-3-ethylcarbodiimide hydrochloride (2.0 g, 10.4 mmol), 1-hydroxy-7-azabenzotriazole (1.4 g, 10.4 mmol) and diisopropylethylamine (3.6 ml, 20.8 mmol) in N,N-dimethylformamide (15 ml) was stirred at room temperature overnight. Water was added and the reaction mixture was extracted with ethyl acetate. The combined organic layers were washed with brine. Drying and solvent evaporation gave 2-tetrazol-1-yl-benzamide; ¹H NMR (CD₃OD, 400 MHz) δ 9.44 (s, 1H), 7.72 (m, 4H).

Step C: Preparation of 2-Tetrazol-1-yl-benzonitrile

To a solution of 2-tetrazol-1-yl-benzamide (1.5 g, 7.9 mmol) in tetrahydrofuran (50 ml) was added (methoxycarbonylsulfamoyl)ammonium hydroxide, inner salt (2.8 g, 11.8 mmol) in three portions over 1.5 h. Water was added and the reaction mixture was extracted with ethyl acetate. The combined organic layers were washed with brine. Drying and solvent evaporation gave 2-tetrazol-1-yl-benzonitrile; ¹H NMR (CDCl₃, 400 MHz) δ 9.27 (s, 1H), 7.90 (m, 3H), 7.72 (m, 1H).

Step D: Preparation of 2-Tetrazol-1-yl-benzylamine

A solution of 2-tetrazol-1-yl-benzonitrile (1.3 g, 7.6 mmol) in ethanol saturated with ammonia (125 ml) was stirred in the presence of Raney nickel (50% slurry in water, washed with ethanol, catalytic amount) under a hydrogen atmosphere overnight. The reaction mixture was filtered over celite and concentrated to give 2-tetrazol-1-yl-benzylamine; ¹H NMR (CDCl₃, 400 MHz) δ 9.28 (s, 1H), 7.59 (m, 2H), 7.47 (m, 2H), 3.70 (s, 2H).

### EXAMPLE IV

5-Chloro-2-tetrazol-1-yl-benzylamine

Prepared following a similar protocol as described in Example III, 2-amino-5-chloro-benzoic acid was converted to 5-chloro-2-tetrazol-1-yl-benzylamine.

¹H NMR (CDCl₃, 400 MHz) δ 9.24 (s, 1H), 7.64 (d, 1H, J=2.2 Hz), 7.46 (m, 1H), 7.38 (m, 1H), 3.68 (s, 2H).

### EXAMPLE V

C-(3-[1,2,4]Triazol-1-yl-pyridin-2-yl)-methylamine Hydrochloride Salt

Step A: Preparation of 3-[1,2,4]Triazol-1-yl-pyridine-2-carbonitrile

To a solution of 2-cyano-3-fluoro-pyridine (2.99 g, 24.49 mmol, preparation described in Sakamoto et.al., Chem. Pharm. Bull. 1985, 33(2), 565-571) in DMF (30 ml) is added cesium carbonate (2.03 g, 29.39 mmol) and 1,2,4-triazole (2.03 g, 29.39 mmol) and the reaction mixture is stirred at 65° C. for 4 h. After cooling to room temperature, the mixture is diluted with water and extracted with EtOAc 3 times. The aqueous layer is saturated with LiCl and further extracted with EtOAc. The combined organic layer is dried on sodium sulfate, concentrated in vacuo. The crude product is purified by flash chromatography (silica gel, 2% MeOH containing 10% NH₄OH in CH₂Cl₂ to 6%) to give 3-[1,2,4]triazol-1-yl-pyridine-2-carbonitrile. ¹H NMR (CDCl₃, 400 MHz) δ 8.95 ((s, 1H); 8.8 (d, J=4 Hz, 1H); 8.24 (s, 1H); 8.22 (d, J=8.5 Hz, 1H); 7.75 (dd, J=4, 8.5 Hz, 1H).

Step B: Preparation of (3-[1,2,4]Triazol-1-yl-pyridin-2-ylmethyl)-carbamic Acid Tert-Butyl Ester

To a suspension of Raney Nickel (ca. 3 pipets of suspension in water, washed/decanted with EtOH several times) in MeOH saturated with NH₃ (200 ml) was added 3-[1,2,4]triazol-1-yl-pyridine-2-carbonitrile (3.745 g, 21.88 mmol). The mixture was hydrogenated at 55 Psi for 18 h. The reaction mixture was filtered on celite under a flow of argon and the filtrate was concentrated in vacuo. To a solution of the crude material in CH₂Cl₂ (100 ml) and MeOH (10 ml) was added di-tert-butyl dicarbonate (6.2 g, 28.4 mmol) and the reaction mixture was stirred at room temperature for 30 min. The crude product obtained by concentration in vacuo is purified by flash chromatography (silica gel, 2% MeOH containing 10% NH₄OH in CH₂Cl₂ to 6%) to give (3-[1,2,4]triazol-1-yl-pyridin-2-ylmethyl)-carbamic acid tert-butyl ester. ¹H NMR (CDCl₃, 400 MHz) δ 8.72 (d, J=4.8 Hz, 1H); 8.42 (s, 1H); 8.18 (s, 1H); 7.70 (d, J=7.6 Hz, 1H); 7.40 (dd, J=4.8, 7.6 Hz, 1H); 5.85 (bs, 1H); 4.43 (d, J=5.4 Hz, 2H); 1.45 (s, 9H).

Step C: Preparation of C-(3-[1,2,4]Triazol-1-yl-pyridin-2-yl)-methylamine Hydrochloride Salt

Through a solution of (3-[1,2,4]triazol-1-yl-pyridin-2-ylmethyl)-carbamic acid tert-butyl ester (4.08 g) in CH₂Cl₂ (100 ml) and MeOH (20 ml) cooled to 0° C. was bubbled HCl (g) for 10 min. The flask was sealed and the reaction mixture was stirred at room temperature for 18 h. Nitrogen was bubbled through the reaction mixture for 5 min and the reaction mixture was concentrated to give C-(3-[1,2,4]triazol-1-yl-pyridin-2-yl)-methylamine hydrochloride salt as a white solid. ¹H NMR (CD₃OD, 400 MHz) δ 9.67 ((s, 1H); 8.85 (d, J=5.3 Hz, 1H); 8.72 ((s, 1H); 8.18 (d, J=8 Hz, 1H); 7.7 (dd, J=5.3, 8 Hz, 1H); 4.45 (s, 2H).

### EXAMPLE VI

tert-Butyl 2-(aminomethyl)benzylcarbamate

Step A: Preparation of 2-(Azidomethyl)benzonitrile:

To a solution of 90 g (459 mmol) 2-cyanobenzylbromide in 600 mL THF was added in one portion a solution of 36 g (553 mmol) sodium azide in 100 mL water. The two phase mixture was stirred at 23° C. for 18 hr. The THF layer was separated from the lower water layer and used in the next step without further purification.

Step B: Preparation of Tert-Butyl 2-cyanobenzylcarbamate:

To the THF layer from the previous step was diluted to a volume of 1.6 L, divided into two equal portions and each hydrogenated at 45 psi in a Parr pressure bottle containing 6 g of 5% palladium on carbon (50% water by weight). A 5-10° C. exotherm was observed within 30 min and shaking continued a total of 1.5 hr. The individual batches were filtered through celite, washed 2×with 100 mL fresh THF and the filtrates combined into a single portion. To the amine mixture without concentration (Caution: attempts to concentrate the solution resulted in a large exotherm and the batch turned black) was added 87.5 mL (381 mmol) of di-tert-butyl dicarbonate neat. After 2 hr the THF was removed in vacuo and flushed with 250 mL of 15% ethyl acetate in hexane. The semi-solid was slurried in 250 mL of 15% ethyl acetate and filtered. The filtrate was concentrated in vacuo, diluted with 10% ethyl acetate in hexane (175 mL), cooled to 0° C. and filtered to give tert-butyl 2-cyanobenzylcarbamate as a gray solid.

Step C: Preparation of Tert-Butyl 2-(aminomethyl)benzylcarbamate Hemisulfate:

To a 3 L, 3 neck flask fitted with a thermocouple, a condenser and nitrogen inlet was added 3 g (23 mmol) of cobaltous chloride, then 1200 mL of THF followed by 59 g (254 mmol) of tert-butyl 2-cyanobenzylcarbamate and 600 mL of ice-water. To the light pink solution at 15° C. was added 26 g (684 mmol) of sodium borohydride in portions as follows. The initial 3 g of sodium borohydride resulted in a vigorous hydrogen gas evolution and formation of a black suspension. The batch temperature reached 35° C. within 2 hr, and was maintained at this temperature with a heating mantle. Additional sodium borohydride and cobaltous chloride were added as needed to drive the reaction to completion. Typically, 2×7.5 g of additional sodium borohydride and 2×1 g portions of cobaltous chloride were added at 12 hour intervals. Once complete, the layers were allowed to settle and the clear upper THF layer was decanted from the black aqueous layer. The aqueous layer was washed with 750 mL fresh THF, the two THF layers combined and filtered through a pad of celite. The orange-yellow filtrate was concentrated to about 300 mL in vacuo, resulting in water layer with the product as an oily lower layer. The mixture was extracted with 2×250 mL ethyl acetate and the combined extracts reacted with 24 g (200 mmol) of solid sodium hydrogensulfate. A solid formed immediately, and the slurry was stirred for 30 min, filtered and washed with 2×100 mL ethyl acetate to give 62 g of a white powder. The powder was slurried in 175 mL water, cooled to 0° C., filtered, washed with 2×40 mL cold water and the solid dried in a vacuum oven at 55° C. for 24 hr to give tert-butyl 2-cyanobenzylcarbamate) of tert-butyl 2-(aminomethyl)benzylcarbamate hemisulfate salt as a white powder.

### EXAMPLE VII

tert-Butyl 2-[2-(aminomethyl)-4-chlorophenyl]ethylcarbamate

Step A: Preparation of 2-(tert-butoxycarbonyl)-7-chloro-3,4-dihydro-2H-isoquinolin-1-one

Into a stirred solution of 7-chloro-3,4-dihydroisoquinolin-1-one (11.6 g, 12.6 mMol) in 50 mL of anhydrous N,N-dimethylformamide under inert atmosphere at ambient temperature was added diisopropylethylamine (17.0 mL, 95.8 mMol, 1.5 eq), di-tert-butyl dicarbonate (15.33 g, 70.26 mMol, 1.1 eq), and a catalytic amount of 4-(dimethylamino)pyridine. This was stirred at ambient temperature for 2 hours, concentrated in vacuo, then partitioned between methylene chloride and water. The organics were dried (Na₂SO₄) and concentrated in vacuo. The residue was purified by flash silica gel chromatography using methylene chloride as an eluent. Desired fractions were concentrated in vacuo to afford 2-(tert-butoxycarbonyl)-7-chloro-3,4-dihydro-2H-isoquinolin-1-one (2) as a clear colorless oil (hplc rt=3.55 min, method A; mass spec m/z=282.1).

Step B: Preparation of 2-(tert-butoxycarbonylaminoethyl)-5-chlorobenzyl Alcohol

Into a stirred solution of 2-(tert-butoxycarbonyl)-7-chloro-3,4-dihydro-2H-isoquinolin-1-one (18.4 g, 64.38 mMol) in 50 mL of anhydrous tetrahydrofuran under inert atmosphere at 0° C. was added 2.0M LiBH₄ in tetrahydrofuran (64.38 mL, 128.76 mMol, 2 eq). This was stirred at 0° C. for 1.5 hours, quenched with saturated ammonium chloride solution, then partitioned between ethyl acetate and water. The organics were dried (Na₂SO₄) and concentrated in vacuo. This afforded 2-(tert-butoxycarbonylaminoethyl)-5-chlorobenzyl alcohol as a clear colorless oil (hplc rt=3.21 min, method A; mass spec m/z=286.2).

Step C: Preparation of tert-Butyl 2-[2-azidomethyl)-4-chlorophenyl]ethylcarbamate:

To a solution of 649 mg (2.27 mmol) tert-butyl 2-[4-chloro-2-(hydroxymethyl)phenyl]-ethylcarbamate in 5.0 mL THF at 0° C. was added 0.674 mL (3.13 mmol) of diphenylphosphoryl azide (DPPA) and 0.468 mL (3.13 mmol) of 1,8-diazabicyclo-[5.4.0]undec-7-ene (DBU) and the reaction stirred at 0° C. for 10 min, then at room temperature. After 3 h the reaction was treated with saturated sodium carbonate (aq) and extracted with ethyl acetate (3×). The organic extracts were combined, washed with brine (1×), dried over sodium sulfate, filtered and concentrated to dryness in vacuo to give 963 mg of a crude oil. Flash chromatography on silica gel (15% ethyl acetate in hexane) gave tert-butyl 2-[2-azidomethyl)-4-chlorophenyl] ethylcarbamate as a colorless oil: ¹H NMR (DMSO-d₆, 400 MHz): δ 7.44 (d, 1H, J=1.8 Hz), 7.36 (dd, 1H, J=2.0 and 8.2 Hz), 7.26 (d, 1H, J=8.1 Hz), 6.92 (br t, 1H, J=5.5 Hz), 4.53 (s, 2H), 3.10 (dt, 2H, J=6.3 and 7.6 Hz), 2.73 (t, 2H, J=7.3 Hz), 1.36 (s, 9H); MS ES: M+Na=333.0; TLC Rf=0.32 (15% ethyl acetate in hexane).

Step D: Preparation of Tert-Butyl 2-[2-(aminomethyl)-4-chlorophenyl]ethylcarbamate:

To a solution of 629 mg (2.02 mmol) tert-butyl 2-[2-azidomethyl)-4-chlorophenyl]ethylcarbamate in 30.0 mL THF containing 3.1 mL water was added 1.06 g (4.05 mmol) triphenylphosphine and the reaction stirred at room temperature overnight. The THF was removed in vacuo and the residual aqueous phase extracted with methylene chloride (3×). The organics were combined, washed with brine (1×), dried over sodium sulfate, filtered and concentrated to dryness in vacuo. Flash chromatography on silica gel (linear gradient from 266/10/1 to 200/10/1 of methylene chloride/methanol/concentrated ammonium hydroxide) gave tert-butyl 2-[2-(aminomethyl)-4-chlorophenyl]-ethylcarbamate as a colorless oil: ¹H NMR (DMSO-d₆, 400 MHz): δ 7.45 (d, 1H, J=1.7 Hz), 7.18 (dd, 1H, J=2.1 and 8.2 Hz), 7.12 (d, 1H, J=8.2 Hz), 7.01 (br t, 1H, J=5.2 Hz), 3.73 (s, 2H), 3.07 (dt, 2H, J=6.5 and 7.3 Hz), 2.68 (t, 2H, J=7.4 Hz), 1.36 (s, 9H); MS ES: M+H=285.1; TLC R_{f}=0.33 (160/10/1 of methylene chloride/methanol/concentrated ammonium hydroxide).

### EXAMPLE VIII

5-Chloro-2-[1,2,4]triazol-1-yl-benzylamine

Step A: Preparation of 5-Chloro-2-[1,2,4]triazol-1-yl-benzonitrile

To a solution of 2,5-dichlorobenzonitrile (10 g, 58.1 mmol) in DMF (100 ml) was added cesium carbonate (22.7 g, 69.8 mmol) and 1,2,4-triazole (4.8 g, 69.8 mmol) and the reaction mixture was stirred at 65° C. for 5.5 h, at 75° C. for 16 h, at 85° C. for 7 h. More 1,2,4-triazole (5 g) was added and the reaction mixture was stirred at 85° C. for 18 h and at 100° C. for 4 h. After cooling to room temperature, the mixture was diluted with water and extracted with EtOAc 3 times. The combined organic layer was washed with aqueous LiCl, dried on sodium sulfate, concentrated in vacuo to give 5-chloro-2-[1,2,4]triazol-1-yl-benzonitrile as a white solid which was used in the next step without further purification.

Step B: Preparation of 5-Chloro-2-[1,2,4]triazol-1-yl-benzylamine

To a suspension of 5-chloro-2-[1,2,4]triazol-1-yl-benzonitrile (11.87 g, 58 mmol) in EtOH saturated with NH₃ (500 ml) was added Raney Nickel (ca. 5 pipets of suspension in water, washed/decanted with EtOH several times). The mixture was hydrogenated at 1 atm for 26 h. The reaction mixture was filtered on celite under a flow of argon and the filtrate was concentrated in vacuo. The crude product was purified by flash chromatography (silica gel, 5% MeOH containing 10% NH₄OH in CH₂Cl₂ to 10%) to give 5-chloro-2-[1,2,4]triazol-1-yl-benzylamine as a white solid.

¹H NMR (CDCl₃, 400 MHz) δ 8.47 (s, 1H); 8.14 (s, 1H); 7.58 (d, J=2.3 Hz, 1H); 7.38 (dd, J=2.3, 7.9 Hz, 1H); 7.30 (d, J=7.9 Hz, 1H); 3.70 (s, 2H).

### EXAMPLE IX

2-(1,2,4-Triazol-1-yl)benzylamine

Step A: Preparation of 2-(1,2,4-Triazol-1-yl)cyanobenzene

To a stirred solution of 2-fluorocyanobenzene (5.0 g, 41 mmol) in DMF (75 mL) was added 1,2,4-triazole (3.0 g, 43 mmol) and cesium carbonate (14 g, 43 mmol). The mixture was warmed to 50° C. and stirred under inert atmosphere for 18 h. The mixture was cooled to ambient temperature, diluted with an equal volume of EtOAc, filtered, and the filtrate solvents were removed under reduced pressure. The residue was partitioned between ether (50 mL) and water (100 mL). The undissolved solid was collected by suction filtration and dried under reduced pressure to give 4.6 g of a 10:1 mixture of 2-(1,2,4-triazol-1-yl)cyanobenzene (hplc retention time=2.29 min, 5% to 100% CH₃CN in water containing 0.1% TFA, Zorbax C8, 4.6 mm ID×7.5 cm, 3.5 micron; TLC Rf=0.6, EtOAc) and 2-(1,2,4-triazol-4-yl)cyanobenzene (hplc retention time=1.91 min, 5% to 100% CH₃CN in water containing 0.1% TFA, Zorbax C8, 4.6 mm ID×7.5 cm, 3.5 micron; TLC Rf=0.1, EtOAc). The mixture was separated by flash chromatography using a gradient elution of 0:100 to 5:95 MeOH:EtOAc to give 2-(1,2,4-triazol-1-yl)cyanobenzene (¹H NMR (DMSO-d₆) δ 9.19 (s, 1H), 8.37 (s, 1H), 8.10 (d, J=7.6 Hz, 1H), 7.96-7.87 (m, 2H), 7.71 (t, J=7.7 Hz, 1H); mass spec m/z=171 (M⁺+H)) and 2-(1,2,4-triazol-4-yl)cyanobenzene (¹H NMR (DMSO-d₆) δ 9.03 (s, 2H), 8.13 (d, J=7.6 Hz, 1H), 7.93(t, J=7.8 Hz, 1H), 7.80 (d, J=7.8 Hz, 1H), 7.74 (t, J=7.7 Hz, 1H); mass spec m/z=171 (M⁺+H)), both as white solids.

Step B: Preparation of 2-(1,2,4-Triazol-1-yl)-benzylamine

A solution of 2-(1,2,4-triazol-1-yl)cyanobenzene (508 mg, 2.99 mmol) and 25% by weight of palladium on carbon, 10% catalyst (134 mg) in ethanol (75 ml) was placed on a PARR Hydrogenation Apparatus under a hydrogen atmosphere at 55 psi. overnight. The mixture was filtered through celite and concentrated to give 2-(1,2,4-triazol-1-yl)-benzylamine; ¹H NMR (CD₃OD) δ 8.80 (0 (s, 1H), 8.22 (s, 1H), 7.64-7.43 (m, 4H), 3.66 (s, 2H).

### EXAMPLE 1a

2-[3-cyano-6-(2,2-difluoro-2-pyridin-2-yl-ethylamino)-pyridin-2-yl]-N-(2,5-dichloro-benzyl)-acetamide

Step A: Preparation of 2-(6-tert-Butoxycarbonylamino-3-cyano-pyridin-2-yl)-acetic Acid Ethyl Ester

To a suspension of 2-methyl-3-cyano-6-amino-pyridine (20 g, 150.2 mmol, Tetrahedron Lett., 1999, 40(47), 8193-8195) in dichloromethane (200 mL) at room temperature was added triethylamine (23.0 mL, 165.2 mmol) and di-tert-Butyl dicarbonate (34.42 g, 157.7 mmol). After stirring for 4 days the reaction mixture was concentrated in vacuo and purified by flash chromatography (silica gel, 15% EtOAc in hexane to 30%) to give 2-methyl-3-cyano-6-tert-Butoxycarbonylamino-pyridine as a white solid. ¹H NMR (CDCl₃, 400 MHz): δ 7.88 (A of AB, d, J=9 Hz, 1H); 7.82 (B of AB, d, J=9 Hz, 1H); 7.42 (b s, 1H); 2.65 (s, 3H); 1.50 (s, 9H).

To a solution of 2-methyl-3-cyano-6-tert-Butoxycarbonylamino-pyridine (34 g, 145.8 mmol) in THF (300 ml) cooled to −78° C. was added lithium diisopropyl amide (189.5 ml of a 2 M solution in heptane/THF/ethylbenzene, 378.96 mmol) dropwise. After stirring at −78° C. for 15 min, diethyl carbonate (44.15 ml, 364.4 mmol) dropwise. The reaction mixture was stirred at −78° C. for 30 min and allowed to warm to room temperature. The reaction mixture was concentrated to ⅔ volume in vacuo, diluted with diethyl ether, washed with aqueous ammonium chloride, with brine, dried on MgSO₄, concentrated in vacuo and purified by flash chromatography (silica gel, 15% EtOAc in hexane to 25%) to give 2-(6-tert-Butoxycarbonylamino-3-cyano-pyridin-2-yl)-acetic acid ethyl ester as a yellow syrup which crystallizes over time. ¹H NMR (CDCl₃, 400 MHz): δ 7.89 (A of AB, d, J=9 Hz, 1H); 7.84 (B of AB, d, J=9 Hz, 1H); 7.41 (b s, 1H); 4.20 (q, J=7 Hz, 2H); 3.94 (s, 2H); 1.52 (s, 9H); 1.20 (t, J=7 Hz, 3H).

Step B: Preparation of 2-[3-cyano-6-(2,2-difluoro-2-pyridin-2-yl-ethyl-tertButoxycarbonylamino)-pyridin-2-yl]-acetic Acid

To a solution of 2-(6-tert-Butoxycarbonylamino-3-cyano-pyridin-2-yl)-acetic acid ethyl ester (33 g, 108.1 mmol) in DMF (300 ml) cooled to 0° C. was added 1,1,1-trifuoro-methanesulfonic acid 2,2-difluoro-2-pyridin-2-yl-ethyl ester (20.6 g, 70.88 mmol, Patent Publication WO 00/75134) dropwise, followed by NaH (5.4 g, 135 mmol, 60% in oil) by portions. The reaction mixture was stirred at 0° C. for 10 min and allowed to warm to room temperature. The reaction mixture is stirred at room temperature for 5 hours and 30 minutes, diluted with diethyl ether and ethyl acetate, washed with water, with aqueous sodium bicarbonate and with aqueous LiCl. The organic layer is dried on Na₂SO₄, concentrated in vacuo and purified by flash chromatography (silica gel, 10% EtOAc in hexane to 40%) to give 2-(6-tert-Butoxycarbonylamino-3-cyano-pyridin-2-yl)-acetic acid ethyl ester (15 g recovered SM) and 2-[3-cyano-6-(2,2-difluoro-2-pyridin-2-yl-ethyl-tertButoxycarbonylamino)-pyridin-2-yl]-acetic acid ethyl ester. ¹H NMR (CDCl₃, 400 MHz): δ 8.62 (d, J=4.5 Hz, 1H); 7.82 (s, 2H); 7.76 (t, J=7.5 Hz, 1H); 7.55 (d, J=7.5 Hz, 1H); 7.35 (dd, J=4.5, 7.5 Hz, 1H); 5.05 (t, J=13.9 Hz, 2H); 4.18 (q, J=7.5 Hz, 2H); 3.70 (s, 2H); 1.48 (s, 9H); 1.24 (t, J=7.5 Hz, 3H).

To a solution of 2-[3-cyano-6-(2,2-difluoro-2-pyridin-2-yl-ethyl-tertButoxycarbonylamino)-pyridin-2-yl]-acetic acid ethyl ester (5 g, 11.2 mmol) in THF (80 ml) was added 1N LiOH (22.4 ml, 22.4 mmol). The reaction mixture was stirred at room temperature vigorously for 4 h, 1N HCl (22.4 ml, 22.4 mmol) was added. The THF was removed by concentration in vacuo and the aqueous residue was extracted with dichloromethane (×3). The combined organic layer was dried on Na₂SO₄ and concentrated in vacuo to give 2-[3-cyano-6-(2,2-difluoro-2-pyridin-2-yl-ethyl-tertButoxycarbonylamino)-pyridin-2-yl]-acetic acid as a pale yellow foam. ¹H NMR (CDCl₃, 400 MHz): δ 8.72 (d, J=4.8 Hz, 1H); 7.93 (A of AB, d, J=9 Hz, 1H);); 7.84 (B of AB, d, J=9 Hz, 1H); 7.82 (t, J=7 Hz, 1H); 7.58 (d, J=7 Hz, 1H); 7.41 (dd, J=4.8, 7 Hz, 1H); 5.10 (t, J=14.8 Hz, 2H); 4.00 (s, 2H);); 1.52 (s, 9H).

MS ES+M+1=419.5, M+Na=441.4.

Step C: 2-[3-cyano-6-(2,2-difluoro-2-pyridin-2-yl-ethylamino)-pyridin-2-yl]-N-(2,5-dichloro-benzyl)-acetamide

To a solution of 2-[3-cyano-6-(2,2-difluoro-2-pyridin-2-yl-ethyl-tertButoxycarbonylamino)-pyridin-2-yl]-acetic acid (100 mg, 0.20 mmol, material containing 2 eq LiCl) in DMF (3 ml) is added 2,5-dichloro-benzyl amine (35 mg, 0.2 mmol), 1-hydroxy-7-azabenzotriazole (29.8 mg, 0.22 mmol) and 1-ethyl-3-(3-dimethylaminopropyl)carbodiimide (49.6 mg, 0.26 mmol) and the reaction mixture is stirred at room temperature for 16 h. The reaction mixture is diluted with ethyl acetate, washed with water and with aqueous LiCl. The organic layer is dried on Na₂SO₄, concentrated in vacuo and purified by flash chromatography (silica gel, 1% MeOH (containing 10% NH₄OH) in dichloromethane to 4%) to give {5-cyano-6-[(2,5-dichloro-benzylcarbamoyl)-methyl]-pyridin-2-yl}-(2,2-difuoro-2-pyridin-2-yl-ethyl)-carbamic acid dimethyl-ethyl ester which is used directly in the next step.

A solution of {5-cyano-6-[(2,5-dichloro-benzylcarbamoyl)-methyl]-pyridin-2-yl}-(2,2-difuoro-2-pyridin-2-yl-ethyl)-carbamic acid dimethyl-ethyl ester (50 mg, 0.087 mmol) in anhydrous dichloromethane (3 ml) and anhydrous trifluoroacetic acid (1 ml) was allowed to stand at room temperature under argon for 1 h. The reaction was concentrated in vacuo and azeothroped with toluene twice. The crude residue was purified by reverse phase preparative HPLC (5% to 95% CH₃CN in water containing 0.1% TFA, C18 PRO YMC 20×150 mm) to provide 2-[3-cyano-6-(2,2-difluoro-2-pyridin-2-yl-ethylamino)-pyridin-2-yl]-N-(2,5-dichloro-benzyl)-acetamide as its bis-TFA salt. ¹H NMR (CD₃OD, 400 MHz): δ 8.70 (b s, 1H); 8.62-8.54 (m, 1H); 7.94-7.84 (m, 1H); 7.70-7.62 (m, 1H); 7.62-7.53 (m, 1H); 7.50-7.42 (m, 1H); 7.48-7.30 (m, 2H); 7.28-7.20 (m, 1H); 6.56-6.48 (m, 1H); 4.48 (s, 2H); 4.33 (t, J=14.8 Hz, 2H); 3.78 (s, 2H).

MS ES+M+1=476.3.

### EXAMPLE 1b

2-[3-cyano-6-(2,2-difluoro-2-pyridin-2-yl-ethylamino)-1-oxy-pyridin-2-yl]-N-(2,5-dichloro-benzyl)-acetamide and 2-{3-cyano-6-[2,2-difluoro-2-(1-oxy-pyridin-2-yl)-ethylamino]-1-oxy-pyridin-2-yl}-N-(2,5-dichloro-benzyl)-acetamide

To a solution of 2-[3-cyano-6-(2,2-difluoro-2-pyridin-2-yl-ethylamino)-pyridin-2-yl]-N-(2,5-dichloro-benzyl)-acetamide (10 mg, 0.02 mmol) in dichloroethane (2 ml) was added m-CPBA (7 mg, 0.03 mmol, 70-75%). The reaction mixture is stirred at 60° C. and followed by LC/MS. After 1 h, the reaction mixture is allowed to cool to room temperature and the dichloroethane is removed by blowing nitrogen over the sample. The crude residue is purified by reverse phase preparative HPLC (5% to 95% CH₃CN in water containing 0.1% TFA, C18 PRO YMC 20×150 mm) to provide 2-[3-cyano-6-(2,2-difluoro-2-pyridin-2-yl-ethylamino)-1-oxy-pyridin-2-yl]-N-(2,5-dichloro-benzyl)-acetamide as TFA salt: ¹H NMR (CDCl₃, 400 MHz): δ 8.68 (d, J=4.1 Hz, 1H); 7.97-7.90 (m, 1H); 7.90-7.83 (m, 1H); 7.71 (d, J=7.8 Hz, 1H); 7.55 (d, J=9.3 Hz, 1H); 7.49-7.42 (m, 1H); 7.33 (d, J=2.6 Hz, 1H); 7.16 (dd, J=9.3, 2.6 Hz, 1H); 6.96 (d, J=9.3 Hz, 1H); 4.47 (d, J=6.4 Hz, 2H); 4.32 (td, J=13.8, 8.3 Hz, 2H); 4.16 (s, 2H).

MS ES+M+1=492.4.

and 2-{3-cyano-6-[2,2-difluoro-2-(1-oxy-pyridin-2-yl)-ethylamino]-1-oxy-pyridin-2-yl}-N-(2,5-dichloro-benzyl)-acetamide as free base: ¹H NMR (CDCl₃, 400 MHz): δ 8.31 (d, J=6.2 Hz, 1H); 8.02-7.97 (m, 1H); 7.87-7.80 (m, 1H); 7.69 (dd, J=7.8, 2.3 Hz, 1H); 7.52-7.36 (m, 2H); 7.49 (d, J=9.3 Hz, 1H); 7.31 (d, J=2.8 Hz, 1H); 7.20-7.14 (m, 2H); 4.64 (td, J=14.4, 8.1 Hz, 2H); 4.45 (d, J=6.5 Hz, 1H); 4.17 (s, 2H).

MS ES+M+1=508.4.

Alternatively, monoxidation can be performed with 1 eq mCPBA at 40° C. to obtain the mono-N-oxide, and then in a second step the mono-N-oxide can be exposed to excess mCPBA at 60° C. to obtain the bis N-oxide.

### EXAMPLE 2a

2-[3-cyano-6-(2,2-difluoro-2-pyridin-2-yl-ethylamino)-pyridin-2-yl]-N-(2-aminomethyl-5-chloro-benzyl)-acetamide

To a solution of 2-[3-cyano-6-(2,2-difluoro-2-pyridin-2-yl-ethyl-tert-butoxycarbonylamino)-pyridin-2-yl]-acetic acid (500 mg, 1.19 mmol) in DMF (5 ml) is added 2-tert-butoxycarbonylaminomethyl-5-chloro-benzyl amine (480 mg, 1.78 mmol, example Ia), 1-hydroxy-7-azabenzotriazole (162 mg, 1.19 mmol) and 1-ethyl-3-(3-dimethylaminopropyl)carbodiimide (342 mg, 1.78 mmol) and the reaction mixture is stirred at room temperature for 16 h. The reaction mixture is diluted with ethyl acetate, washed with water and with aqueous LiCl. The organic layer is dried on Na₂SO₄, concentrated in vacuo and purified by flash chromatography (silica gel, 1% MeOH (containing 10% NH₄OH) in dichloromethane to 4%) to give {5-cyano-6-[(2-tertButoxycarbonylaminomethyl-5-chloro-benzylcarbamoyl)-methyl]-pyridin-2-yl}-(2,2-difuoro-2-pyridin-2-yl-ethyl)-carbamic acid dimethyl-ethyl ester, MS ES+M+1=671.4, which is used directly in the next step.

A solution of {5-cyano-6-[(2-tertButoxycarbonylaminomethyl-5-chloro-benzylcarbamoyl)-methyl]-pyridin-2-yl}-(2,2-difuoro-2-pyridin-2-yl-ethyl)-carbamic acid dimethyl-ethyl ester (800 mg, 1.19 mmol) in anhydrous dichloromethane (3 ml) and anhydrous trifluoroacetic acid (1 ml) was allowed to stand at room temperature under argon for 20 min. The reaction was concentrated in vacuo and azeothroped with toluene twice. The crude residue was purified by reverse phase preparative HPLC (5% to 95% CH₃CN in water containing 0.1% TFA, C18 PRO YMC 20×150 mm) to provide 2-[3-cyano-6-(2,2-difluoro-2-pyridin-2-yl-ethylamino)-pyridin-2-yl]-N-(2,5-dichloro-benzyl)-acetamide as its bis-TFA salt. ¹H NMR (CD₃OD, 400 MHz): δ 8.55 (d, J=4.8 Hz, 1H); 7.85-7.83 (m, 1H); 7.63-7.61 (m, 1H); 7.49 (d, J=8.8 Hz, 1H); 7.55-7.35 (m, 3H); 7.35-7.25 (m, 2H); 6.43 (d, J=8.8 Hz, 1H); 4.38 (s, 2H); 4.25 (t, J=15.2 Hz, 2H); 4.18 (s, 2H); 3.63 (s, 2H). MS ES+M+1=471.5.

### EXAMPLE 2b

2-[3-cyano-6-(2,2-difluoro-2-pyridin-2-yl-ethylamino)-1-oxy-pyridin-2-yl]-N-(2-aminomethyl-5-chloro-benzyl)-acetamide

To a solution of 2-[3-cyano-6-(2,2-difluoro-2-pyridin-2-yl-ethylamino)-pyridin-2-yl]-N-(2-aminomethyl-5-chloro-benzyl)-acetamide (503 mg, 1.07 mmol) in dichloromethane (18 ml) is added triethyl amine (0.97 ml, 13.2 mmol) and di-tertButyl-dicarbonate (210 mg, 0.96 mmol), and the reaction mixture is stirred at room temperature for 30 min. The reaction mixture is diluted with dichloromethane, washed with aqueous sodium bicarbonate, dried on Na₂SO₄, concentrated in vacuo and purified by flash chromatography (silica gel, 1% MeOH (containing 10% NH₄OH) in dichloromethane to 4%) to give 2-[3-cyano-6-(2,2-difluoro-2-pyridin-2-yl-ethylamino)-pyridin-2-yl]-N-(2-tertButoxycarbonylaminomethyl-5-chloro-benzyl)-acetamide (450 mg), which is used directly in the next step.

To a solution of 2-[3-cyano-6-(2,2-difluoro-2-pyridin-2-yl-ethylamino)-pyridin-2-yl]-N-(2-tertButoxycarbonylaminomethyl-5-chloro-benzyl)-acetamide (450 mg, 0.79 mmol) in dichloroethane (10 ml) is added mCPBA (194 mg, 0.79 mmol, 70%) and the reaction mixture is stirred at 50° C. for 1 h. The reaction mixture is diluted with dichloromethane, washed with 1M Na₂SO₃, with aqueous sodium bicarbonate, dried on Na₂SO₄, concentrated in vacuo and purified by flash chromatography (silica gel, 1% MeOH (containing 10% NH₄OH) in dichloromethane to 4%) to give 2-[3-cyano-6-(2,2-difluoro-2-pyridin-2-yl-ethylamino)-1-oxy-pyridin-2-yl]-N-(2-tertButoxycarbonylaminomethyl-5-chloro-benzyl)-acetamide, MS ES+M+1=587.1, which is used directly in the next step.

A solution of 2-[3-cyano-6-(2,2-difluoro-2-pyridin-2-yl-ethylamino)-1-oxy-pyridin-2-yl]-N-(2-tertButoxycarbonylaminomethyl-5-chloro-benzyl)-acetamide in anhydrous trifluoroacetic acid (2 ml) was allowed to stand at room temperature under argon for 20 min. The reaction was concentrated in vacuo. The crude residue was purified by reverse phase preparative HPLC (5% to 95% CH₃CN in water containing 0.1% TFA, C18 PRO YMC 20×150 mm) to provide 2-[3-cyano-6-(2,2-difluoro-2-pyridin-2-yl-ethylamino)-1-oxy-pyridin-2-yl]-N-(2-aminomethyl-5-chloro-benzyl)-acetamide as bis TFA salt. ¹H NMR (CD₃OD, 400 MHz): δ 8.67 (d, J=4.2 Hz, 1H); 8.02-7.92 (m, 1H); 7.75 (d, J=8.1 Hz, 1H); 7.63 (d, J=9.2 Hz, 1H); 7.52-7.50 (m, 2H); 7.45-7.35 (m, 2H); 7.12 (d, J=9.2 Hz, 1H); 4.48 (s, 2H); 4.35 (t, J=14 Hz, 2H); 4.25 (s, 2H); 4.02 (s, 2H).

MS ES+M+1=487.4.

### EXAMPLE 2c

2-{3-cyano-6-[2,2-difluoro-2-(1-oxy-pyridin-2-yl)-ethylamino]-1-oxy-pyridin-2-yl}-N-(2-aminomethyl-5-chloro-benzyl)-acetamide

To a solution of 2-[3-cyano-6-(2,2-difluoro-2-pyridin-2-yl-ethylamino)-1-oxy-pyridin-2-yl]-N-(2-tertButoxycarbonylaminomethyl-5-chloro-benzyl)-acetamide (250 mg, 0.43 mmol) in dichloroethane (7.5 ml) is added mCPBA (105 mg, 0.43 mmol, 70%) and the reaction mixture is stirred at 65° C. for 1.5 h. Two additional portions of mCPBA (140 mg and 48 mg) were added in the next 30 min, until reaction completion. The reaction mixture is diluted with dichloromethane, washed with 1M Na₂SO₃, with aqueous sodium bicarbonate, dried on Na₂SO₄, concentrated in vacuo to give crude 2-{3-cyano-6-[2,2-difluoro-2-(1-oxy-pyridin-2-yl)-ethylamino]-1-oxy-pyridin-2-yl}-N-(2-tertButoxycarbonylaminomethyl-5-chloro-benzyl)-acetamide which is used directly in the next step.

A solution of 2-{3-cyano-6-[2,2-difluoro-2-(1-oxy-pyridin-2-yl)-ethylamino]-1-oxy-pyridin-2-yl}-N-(2-tertButoxycarbonylaminomethyl-5-chloro-benzyl)-acetamide in anhydrous trifluoroacetic acid (1 ml) was allowed to stand at room temperature under argon for 20 min. The reaction was concentrated in vacuo. The crude residue was purified by reverse phase preparative HPLC (5% to 95% CH₃CN in water containing 0.1% TFA, C 18 PRO YMC 20×150 mm) to provide 2-{3-cyano-6-[2,2-difluoro-2-(1-oxy-pyridin-2-yl)-ethylamino]-1-oxy-pyridin-2-yl}-N-(2-aminomethyl-5-chloro-benzyl)-acetamide as TFA salt. ¹H NMR (CD₃OD, 400 MHz): δ 8.43 (d, J=6.3 Hz, 1H); 7.85-7.75 (m, 1H); 7.70-7.35 (m, 6H); 7.26 (d, J=9 Hz, 1H); 4.62 (t, J=14 Hz, 2H); 4.45 (s, 2H); 4.03 (s, 2H).

MS ES+M+1=503.4.

### EXAMPLE 3a

2-[3-cyano-6-(2,2-difluoro-2-pyridin-2-yl-ethylamino)-pyridin-2-yl]-N-(3-chloro-benzyl)-acetamide

2-[3-cyano-6-(2,2-difluoro-2-pyridin-2-yl-ethylamino)-pyridin-2-yl]-N-(3-chloro-benzyl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 3-chloro-benzyl amine, using a similar procedure as described in example 1a, step C.

MS ES+M+1=442.5.

### EXAMPLE 3b

2-[3-cyano-6-(2,2-difluoro-2-pyridin-2-yl-ethylamino)-1-oxy-pyridin-2-yl]-N-(3-chloro-benzyl)-acetamide

2-[3-cyano-6-(2,2-difluoro-2-pyridin-2-yl-ethylamino)-1-oxy-pyridin-2-yl]-N-(3-chloro-benzyl)-acetamide was prepared from 2-[3-cyano-6-(2,2-difluoro-2-pyridin-2-yl-ethylamino)-pyridin-2-yl]-N-(3-chloro-benzyl)-acetamide using a similar procedure as described in example 1b.

MS ES+M+1=458.4.

### EXAMPLE 4a

2-[3-cyano-6-(2,2-difluoro-2-pyridin-2-yl-ethylamino)-pyridin-2-yl]-N-(2-fluoro-benzyl)-acetamide

2-[3-cyano-6-(2,2-difluoro-2-pyridin-2-yl-ethylamino)-pyridin-2-yl]-N-(2-fluoro-benzyl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 2-fluoro-benzyl amine, using a similar procedure as described in example 1a, step C.

MS ES+M+1=426.5.

### EXAMPLE 4b

2-[3-cyano-6-(2,2-difluoro-2-pyridin-2-yl-ethylamino)-1-oxy-pyridin-2-yl]-N-(2-fluoro-benzyl)-acetamide and 2-{3-cyano-6-[2,2-difluoro-2-(1-oxy-pyridin-2-yl)-ethylamino]-1-oxy-pyridin-2-yl}-N-(2-fluoro-benzyl)-acetamide

2-[3-cyano-6-(2,2-difluoro-2-pyridin-2-yl-ethylamino)-1-oxy-pyridin-2-yl]-N-(2-fluoro-benzyl)-acetamide and 2-{3-cyano-6-[2,2-difluoro-2-(1-oxy-pyridin-2-yl)-ethylamino]-1-oxy-pyridin-2-yl}-N-(2-fluoro-benzyl)-acetamide were prepared from 2-[3-cyano-6-(2,2-difluoro-2-pyridin-2-yl-ethylamino)-pyridin-2-yl]-N-(2-fluoro-benzyl)-acetamide using a similar procedure as described in example 1b.

MS ES+M+1=442.5 and MS ES+M+1=458.5 respectively.

### EXAMPLE 5a

2-[3-cyano-6-(2,2-difluoro-2-pyridin-2-yl-ethylamino)-pyridin-2-yl]-N-(methyl-3-fluoro-pyridin-2-yl)-acetamide

2-[3-cyano-6-(2,2-difluoro-2-pyridin-2-yl-ethylamino)-pyridin-2-yl]-N-(methyl-3-fuoro-pyridin-2-yl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 2-aminomethyl-3-fluoro-pyridine (Patent Publication WO 00/75134), using a similar procedure as described in example 1a, step C.

MS ES+M+1=427.5.

### EXAMPLE 5b

2-[3-cyano-6-(2,2-difluoro-2-pyridin-2-yl-ethylamino)-1-oxy-pyridin-2-yl]-N-(methyl-3-fluoro-pyridin-2-yl)-acetamide

2-[3-cyano-6-(2,2-difluoro-2-pyridin-2-yl-ethylamino)-1-oxy-pyridin-2-yl]-N-(methyl-3-fuoro-pyridin-2-yl)-acetamide was prepared from 2-[3-cyano-6-(2,2-difluoro-2-pyridin-2-yl-ethylamino)-pyridin-2-yl]-N-(methyl-3-fuoro-pyridin-2-yl)-acetamide using a similar procedure as described in example 1b.

MS ES+M+1=443.5.

### EXAMPLE 6

2-[3-cyano-6-(2,2-difluoro-2-pyridin-2-yl-ethylamino)-pyridin-2-yl]-N-(methyl-1,6-naphthyridin-8-yl)-acetamide

2-[3-cyano-6-(2,2-difluoro-2-pyridin-2-yl-ethylamino)-pyridin-2-yl]-N-(methyl-1,6-naphthyridin-8-yl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 8-aminomethyl-1,6-naphthyridine (example II), using a similar procedure as described in example 1a, step C.

MS ES+M+1=460.5.

### EXAMPLE 7a

2-[3-cyano-6-(2,2-difluoro-2-pyridin-2-yl-ethylamino)-pyridin-2-yl]-N-(5-chloro-2-tetrazol-1-yl-benzyl)-acetamide

2-[3-cyano-6-(2,2-difluoro-2-pyridin-2-yl-ethylamino)-pyridin-2-yl]-N-(5-chloro-2-tetrazol-1-yl-benzyl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 5-chloro-2-(tetrazol-1-yl)-benzyl amine (example IV), using a similar procedure as described in example 1 a, step C.

MS ES+M+1=476.5.

### EXAMPLE 7b

2-[3-cyano-6-(2,2-difluoro-2-pyridin-2-yl-ethylamino)-1-oxy-pyridin-2-yl]-N-(5-chloro-2-tetrazol-1-yl-benzyl)-acetamide

2-[3-cyano-6-(2,2-difluoro-2-pyridin-2-yl-ethylamino)-1-oxy-pyridin-2-yl]-N-(5-chloro-2-tetrazol-1-yl-benzyl)-acetamide was prepared from 2-[3-cyano-6-(2,2-difluoro-2-pyridin-2-yl-ethylamino)-pyridin-2-yl]-N-(5-chloro-2-tetrazol-1-yl-benzyl)-acetamide using a similar procedure as described in example 1b.

MS ES+M+1=527.4.

### EXAMPLE 8a

2-[3-cyano-6-(2,2-difluoro-2-pyridin-2-yl-ethylamino)-pyridin-2-yl]-N-(3-[1,2,4]triazol-1-yl-pyridin-2-yl-methyl)-acetamide

2-[3-cyano-6-(2,2-difluoro-2-pyridin-2-yl-ethylamino)-pyridin-2-yl]-N-(3-[1,2,4]triazol-1-yl-pyridin-2-yl-methyl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 2-aminomethyl-3-[1,2,4]triazol-1-yl-pyridine (example V), using a similar procedure as described in example 1a, step C.

MS ES+M+1=476.5.

### EXAMPLE 8b

2-[3-cyano-6-(2,2-difluoro-2-pyridin-2-yl-ethylamino)-1-oxy-pyridin-2-yl]-N-(3-[1,2,4]triazol-1-yl-pyridin-2-yl-methyl))-acetamide

2-[3-cyano-6-(2,2-difluoro-2-pyridin-2-yl-ethylamino)-1-oxy-pyridin-2-yl]-N-(3-[1,2,4]triazol-1-yl-pyridin-2-yl-methyl))-acetamide was prepared from 2-[3-cyano-6-(2,2-difluoro-2-pyridin-2-yl-ethylamino)-pyridin-2-yl]-N-(3-[1,2,4]triazol-1-yl-pyridin-2-yl-methyl)-acetamide using a similar procedure as described in example 1b.

MS ES+M+1=492.5.

### EXAMPLE 9a

2-[3-cyano-6-(2,2-difluoro-2-pyridin-2-yl-ethylamino)-pyridin-2-yl]-N-(2-aminomethyl-benzyl)-acetamide

2-[3-cyano-6-(2,2-difluoro-2-pyridin-2-yl-ethylamino)-pyridin-2-yl]-N-(2-aminomethyl-benzyl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 2-tertButoxycarbonylaminomethyl-benzyl amine (example VI), using a similar procedure as described in example 2a.

MS ES+M+1=437.5.

### EXAMPLE 9b

2-[3-cyano-6-(2,2-difluoro-2-pyridin-2-yl-ethylamino)-1-oxy-pyridin-2-yl]-N-(2-aminomethyl-benzyl)-acetamide

2-[3-cyano-6-(2,2-difluoro-2-pyridin-2-yl-ethylamino)-1-oxy-pyridin-2-yl]-N-(2-aminomethyl-benzyl)-acetamide was prepared from 2-[3-cyano-6-(2,2-difluoro-2-pyridin-2-yl-ethylamino)-pyridin-2-yl]-N-(2-aminomethyl-benzyl)-acetamide using a similar procedure as described in example 2b.

MS ES+M+1=453.5.

### EXAMPLE 10a

2-[3-cyano-6-(2,2-difluoro-2-pyridin-2-yl-ethylamino)-pyridin-2-yl]-N-(2-methylaminomethyl-5-chloro-benzyl)-acetamide

2-[3-cyano-6-(2,2-difluoro-2-pyridin-2-yl-ethylamino)-pyridin-2-yl]-N-(2-methylaminomethyl-5-chloro-benzyl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 2-(N,N-tertButoxycarbonyl-methyl)-aminomethyl-5-chloro-benzyl amine (example 1b), using a similar procedure as described in example 2a.

MS ES+M+1=485.5.

### EXAMPLE 10b

2-[3-cyano-6-(2,2-difluoro-2-pyridin-2-yl-ethylamino)-1-oxy-pyridin-2-yl]-N-(2-methylaminomethyl-benzyl)-acetamide

2-[3-cyano-6-(2,2-difluoro-2-pyridin-2-yl-ethylamino)-1-oxy-pyridin-2-yl]-N-(2-methylaminomethyl-benzyl)-acetamide was prepared from 2-[3-cyano-6-(2,2-difluoro-2-pyridin-2-yl-ethylamino)-pyridin-2-yl]-N-(2-methylaminomethyl-5-chloro-benzyl)-acetamide using a similar procedure as described in example 2b.

MS ES+M+1=501.5.

### EXAMPLE 10c

2-{3-cyano-6-[2,2-difluoro-2-(1-oxy-pyridin-2-yl)-ethylamino]-1-oxy-pyridin-2-yl}-N-(2-methylaminomethyl-benzyl)-acetamide

2-{3-cyano-6-[2,2-difluoro-2-(1-oxy-pyridin-2-yl)-ethylamino]-1-oxy-pyridin-2-yl 1-N-(2-methylaminomethyl-benzyl)-acetamide was prepared from 2-[3-cyano-6-(2,2-difluoro-2-pyridin-2-yl-ethylamino)-1-oxy-pyridin-2-yl]-N-[2-N,N-(tertButoxycarbonyl-methyl)-aminomethyl-benzyl]-acetamide using a similar procedure as described in example 2c.

MS ES+M+1=517.4.

### EXAMPLE 11a

2-[3-cyano-6-(2,2-difluoro-2-pyridin-2-yl-ethylamino)-pyridin-2-yl]-N-(2-aminoethyl-5-chloro-benzyl)-acetamide

2-[3-cyano-6-(2,2-difluoro-2-pyridin-2-yl-ethylamino)-pyridin-2-yl]-N-(2-aminoethyl-5-chloro-benzyl)-acetamide was prepared from 2-[3-cyano-6-(2,2-difluoro-2-pyridin-2-yl-ethyl-tertButoxycarbonylamino)-pyridin-2-yl]-acetic acid (example 1a, step B) and 2-tertButoxycarbonylaminoethyl-5-chloro-benzyl amine (example VII), using a similar procedure as described in example 2a.

MS ES+M+1=485.5.

### EXAMPLE 11b

2-[3-cyano-6-(2,2-difluoro-2-pyridin-2-yl-ethylamino)-1-oxy-pyridin-2-yl]-N-(2-aminoethyl-5-chloro-benzyl)-acetamide

2-[3-cyano-6-(2,2-difluoro-2-pyridin-2-yl-ethylamino)-1-oxy-pyridin-2-yl]-N-(2-aminoethyl-5-chloro-benzyl)-acetamide was prepared from 2-[3-cyano-6-(2,2-difluoro-2-pyridin-2-yl-ethylamino)-pyridin-2-yl]-N-(2-aminoethyl-5-chloro-benzyl)-acetamide using a similar procedure as described in example 2b.

MS ES+M+1=502.1.

### EXAMPLE 11c

2-{3-cyano-6-[2,2-difluoro-2-(1-oxy-pyridin-2-yl)-ethylamino]-1-oxy-pyridin-2-yl}-N-(2-aminoethyl-5-chloro-benzyl)-acetamide

2-{3-cyano-6-[2,2-difluoro-2-(1-oxy-pyridin-2-yl)-ethylamino]-1-oxy-pyridin-2-yl}-N-(2-aminoethyl-5-chloro-benzyl)-acetamide was prepared from 2-[3-cyano-6-(2,2-difluoro-2-pyridin-2-yl-ethylamino)-1-oxy-pyridin-2-yl]-N-[2-tertButoxycarbonylaminoethyl-benzyl]-acetamide using a similar procedure as described in example 2c.

MS ES+M+1=517.5.

### EXAMPLE 12a

2-[6-(2,2-difluoro-2-pyridin-2-yl-ethylamino)-pyridin-2-yl]-N-(2-aminomethyl-5-chloro-benzyl)-acetamide

Step A: preparation of 2-[6-(2,2-difluoro-2-pyridin-2-yl-ethyl-tertButoxycarbonylamino)-pyridin-2-yl]-acetic Acid

2-[6-(2,2-difluoro-2-pyridin-2-yl-ethyl-tertButoxycarbonylamino)-pyridin-2-yl]-acetic acid is prepared from 2-methyl-6-amino-pyridine using a similar procedure as described in example 1a, steps A and B.

Step B: Preparation of 2-[6-(2,2-difluoro-2-pyridin-2-yl-ethylamino)-pyridin-2-yl]-N-(2-aminomethyl-5-chloro-benzyl)-acetamide

2-[6-(2,2-difluoro-2-pyridin-2-yl-ethylamino)-pyridin-2-yl]-N-(2-aminomethyl-5-chloro-benzyl)-acetamide was prepared from 2-[6-(2,2-difluoro-2-pyridin-2-yl-ethyl-tertButoxycarbonylamino)-pyridin-2-yl]-acetic acid (example 12a, step A) and 2-tertButoxycarbonylaminomethyl-benzyl amine (example Ia), using a similar procedure as described in example 2a.

MS ES+M+1=446.5.

### EXAMPLE 12b

2-[6-(2,2-difluoro-2-pyridin-2-yl-ethylamino)-1-oxo-pyridin-2-yl]-N-(2-aminomethyl-5-chloro-benzyl)-acetamide

2-[6-(2,2-difluoro-2-pyridin-2-yl-ethylamino)-1-oxo-pyridin-2-yl]-N-(2-aminomethyl-5-chloro-benzyl)-acetamide was prepared from 2-[6-(2,2-difluoro-2-pyridin-2-yl-ethylamino)-pyridin-2-yl]-N-(2-aminomethyl-5-chloro-benzyl)-acetamide using a similar procedure as described in example 2b.

MS ES+M+1=462.5.

### EXAMPLE 13a

2-[6-(2,2-difluoro-2-pyridin-2-yl-ethylamino)-pyridin-2-yl]-N-(5-chloro-2-tetrazol-1-yl-benzyl)-acetamide

2-[6-(2,2-difluoro-2-pyridin-2-yl-ethylamino)-pyridin-2-yl]-N-(5-chloro-2-tetrazol-1-yl-benzyl)-acetamide was prepared from 2-[6-(2,2-difluoro-2-pyridin-2-yl-ethyl-tertButoxycarbonylamino)-pyridin-2-yl]-acetic acid (example 12a, step A) and 5-chloro-2-(tetrazol-1-yl)-benzyl amine (example IV), using a similar procedure as described in example 1a, step C.

MS ES+M+1=485.4.

### EXAMPLE 13b

2-[6-(2,2-difluoro-2-pyridin-2-yl-ethylamino)-1-oxy-pyridin-2-yl]-N-(5-chloro-2-tetrazol-1-yl-benzyl)-acetamide

2-[6-(2,2-difluoro-2-pyridin-2-yl-ethylamino)-1-oxy-pyridin-2-yl]-N-(5-chloro-2-tetrazol-1-yl-benzyl)-acetamide was prepared from 2-[6-(2,2-difluoro-2-pyridin-2-yl-ethylamino)-pyridin-2-yl]-N-(5-chloro-2-tetrazol-1-yl-benzyl)-acetamide using a similar procedure as described in example 1b.

MS ES+M+1=501.4.

### EXAMPLE 14a

2-[6-(2,2-difluoro-2-pyridin-2-yl-ethylamino)-pyridin-2-yl]-N-(5-chloro-2-[1,2,4]triazol-1-yl-benzyl)-acetamide

2-[6-(2,2-difluoro-2-pyridin-2-yl-ethylamino)-pyridin-2-yl]-N-(5-chloro-2-[1,2,4]triazol-1-yl-benzyl)-acetamide was prepared from 2-[6-(2,2-difluoro-2-pyridin-2-yl-ethyl-tertButoxycarbonylamino)-pyridin-2-yl]-acetic acid (example 12a, step A) and 5-chloro-2-[1,2,4]triazol-1-yl-benzyl amine (example VII), using a similar procedure as described in example 1a, step C.

MS ES+M+1=484.4.

### EXAMPLE 14b

2-[6-(2,2-difluoro-2-pyridin-2-yl-ethylamino)-1-oxy-pyridin-2-yl]-N-(5-chloro-2-[1,2,4]triazol-1-yl-benzyl)-acetamide

2-[6-(2,2-difluoro-2-pyridin-2-yl-ethylamino)-1-oxy-pyridin-2-yl]-N-(5-chloro-2-[1,2,4]triazol-1-yl-benzyl)-acetamide was prepared from 2-[6-(2,2-difluoro-2-pyridin-2-yl-ethylamino)-pyridin-2-yl]-N-(5-chloro-2-[1,2,4]triazol-1-yl-benzyl)-acetamide using a similar procedure as described in example 1b.

MS ES+M+1=500.4.

### EXAMPLE 15

2-(6-amino-3-cyano-1-oxy-pyridin-2-yl)-N-(5-chloro-2-tetrazol-1-yl-benzyl)-acetamide

Step A: Preparation of 2-(6-tert-Butoxycarbonylamino-3-cyano-pyridin-2-yl)-acetic Acid

To a solution of 2-(6-tert-Butoxycarbonylamino-3-cyano-pyridin-2-yl)-acetic acid ethyl ester (2.5 g, 8.19 mmol) in THF (60 ml) is added 1N LiOH (16.38 ml, 16.38 mmol) and the reaction mixture is stirred at room temperature for 2 h45.1N HCl (16.38 ml, 16.38 mmol) is added and most of the THF is removed in vacuo. The aqueous residue is extracted with dichloromethane (×3), dried on Na₂SO₄ and concentrated in vacuo to give 2-(6-tert-Butoxycarbonylamino-3-cyano-pyridin-2-yl)-acetic acid as a yellow foam. ¹H NMR (CDCl₃, 400 MHz): δ 8.03 (A of AB, d, J=9 Hz, 1H); 7.90 (B of AB, d, J=9 Hz, 1H); 7.82 (b s, 1H); 4.00 (s, 2H); 1.55 (s, 9H).

Step B: Preparation of 2-(6-amino-3-cyano-1-oxy-pyridin-2-yl)-N-(5-chloro-2-tetrazol-1-yl-benzyl)-acetamide

To a solution of 2-(6-tert-Butoxycarbonylamino-3-cyano-pyridin-2-yl)-acetic acid (100 mg, 0.36 mmol) in DMF (3 ml) is added 5-chloro-2-(tetrazol-1-yl)-benzyl amine (83 mg, 0.4 mmol, example IV), 1-hydroxy-7-azabenzotriazole (54 mg, 0.40 mmol) and 1-ethyl-3-(3-dimethylaminopropyl)carbodiimide (83 mg, 0.43 mmol) and the reaction mixture is stirred at room temperature for 16 h. The reaction mixture is diluted with ethyl acetate, washed with aqueous sodium bicarbonate and with aqueous LiCl. The organic layer is dried on Na₂SO₄, concentrated in vacuo to give 2-(6-tertButoxycarbonylamino-3-cyano-pyridin-2-yl-N-(5-chloro-2-tetrazol-1-yl-benzyl)-acetamide, MS ES+M+1=469.5, M+Na=491.5, which is used as is in the next step.

To a solution of 2-(6-tertButoxycarbonylamino-3-cyano-pyridin-2-yl-N-(5-chloro-2-tetrazol-1-yl-benzyl)-acetamide (96 mg, 0.21 mmol) in dichloroethane (2.5 ml) is added mCPBA (66 mg, 0.27 mmol, 70%) and the reaction mixture is stirred at 60° C. for 2 h. The reaction mixture is diluted with ethyl acetate, washed with 1M Na₂SO₃, with aqueous sodium bicarbonate, dried on Na₂SO₄, concentrated in vacuo and purified flash chromatography (silica gel, 35% EtOAc in hexane to 75%) to give 2-(6-tertButoxycarbonylamino-3-cyano-1-oxy-pyridin-2-yl-N-(5-chloro-2-tetrazol-1-yl-benzyl)-acetamide (65 mg), MS ES+M+1=485.4, which is used as is in the next step.

A solution of 2-(6-tertButoxycarbonylamino-3-cyano-1-oxy-pyridin-2-yl-N-(5-chloro-2-tetrazol-1-yl-benzyl)-acetamide in anhydrous trifluoroacetic acid (1 ml) was allowed to stand at room temperature under argon for 20 min. The reaction was concentrated in vacuo and azeothroped with benzene. The crude residue was purified by reverse phase preparative HPLC (5% to 95% CH₃CN in water containing 0.1% TFA, C18 PRO YMC 20×150 mm) to provide 2-(6-amino-3-cyano-1-oxy-pyridin-2-yl-N-(5-chloro-2-tetrazol-1-yl-benzyl)-acetamide. ¹H NMR (d₆ DMSO, 400 MHz): δ 9.85 (s, 1H); 8.80 (b t, J=5.8 Hz, 1H); 7.78 (s, 1H); 7.82-7.63 (m, 1H); 7.61 (s, 2H); 7.53 (d, J=9.4 Hz, 1H); 6.83 (d, J=9.4 Hz, 1H); 4.15 (d, J=5.8 Hz, 2H); 3.88 (s, 2H).

MS ES+M+1=385.5.

### EXAMPLE 16

2-(6-amino-3-cyano-1-oxy-pyridin-2-yl)-N-(5-chloro-2-aminomethyl-benzyl)-acetamide

2-(6-amino-3-cyano-1-oxy-pyridin-2-yl-N-(5-chloro-2-aminomethyl-benzyl)-acetamide is prepared from 2-(6-tert-Butoxycarbonylamino-3-cyano-pyridin-2-yl)-acetic acid (example 15, step A) and 2-tertButoxycarbonylaminomethyl-5-chloro-benzyl amine using a similar procedure as described in example 15, step B.

MS ES+M+1=346.2.

### EXAMPLE 17

2-(6-amino-3-chloro-1-oxy-pyridin-2-yl)-N-(5-chloro-2-tetrazol-1-yl-benzyl)-acetamide

Step A: Preparation of 2-(6-tert-Butoxycarbonylamino-3-chloro-pyridin-2-yl)-acetic Acid

To a solution of 2-methyl-6-tertButoxycarbonylamino-pyridine (1 g, 4.8 mmol, example 12a, step A) in dichloroethane (9 ml) is added N-chlorosuccinimide (673 mg, 5 mmol) and the reaction mixture is stirred at 80° C. until disappearance of the starting material. The reaction mixture is concentrated in vacuo and purified flash chromatography (silica gel, 5% EtOAc in hexane to 15%) to give 2-methyl-3-chloro-6-tertButoxycarbonylamino-pyridine. ¹H NMR (CD₃OD, 400 MHz): δ 7.70 (d, J=8.8 Hz, 1H); 7.63 (d, J=8.8 Hz, 1H); 2.47 (s, 3H); 1.51 (s, 9H).

Deprotonation with LDA and addition of diethyl carbonate, followed by hydrolysis with 1N LiOH, using a similar procedure as described in example 1, steps A and B, provided 2-(6-tert-Butoxycarbonylamino-3-chloro-pyridin-2-yl)-acetic acid. ¹H NMR (CDCl₃, 400 MHz): δ 7.87 (d, J=9 Hz, 1H); 7.70 (d, J=9 Hz, 1H); 7.33 (b s, 1H); 3.94 (s, 2H); 1.51 (s, 9H).

Step B: Preparation of 2-(6-amino-3-chloro-1-oxy-pyridin-2-yl)-N-(5-chloro-2-tetrazol-1-yl-benzyl)-acetamide

2-(6-amino-3-chloro-1-oxy-pyridin-2-yl)-N-(5-chloro-2-tetrazol-1-yl-benzyl)-acetamide is prepared from 2-(6-tert-Butoxycarbonylamino-3-chloro-pyridin-2-yl)-acetic acid (example 17, step A) and 5-chloro-2-(tetrazol-1-yl)-benzyl amine (example IV) using a similar procedure as described in example 15, step B.

MS ES+M+1=394.4.

### EXAMPLE 18

2-(6-amino-3-chloro-1-oxy-pyridin-2-yl)-N-(5-chloro-2-[1,2,4]triazol-1-yl-benzyl)-acetamide

2-(6-amino-3-chloro-1-oxy-pyridin-2-yl)-N-(5-chloro-2-[1,2,4]triazol-1-yl-benzyl)-acetamide is prepared from 2-(6-tert-Butoxycarbonylamino-3-chloro-pyridin-2-yl)-acetic acid (example 17, step A) and 5-chloro-2-[1,2,4]triazol-1-yl-benzyl amine (example V) using a similar procedure as described in example 15, step B.

MS ES+M+1=393.4.

### EXAMPLE 19

2-(6-amino-3-chloro-1-oxy-pyridin-2-yl)-N-(5-chloro-2-aminomethyl-benzyl)-acetamide

2-(6-amino-3-chloro-1-oxy-pyridin-2-yl)-N-(5-chloro-2-aminomethyl-benzyl)-acetamide is prepared from 2-(6-tert-Butoxycarbonylamino-3-chloro-pyridin-2-yl)-acetic acid (example 17, step A) and 2-tertButoxycarbonylaminomethyl-5-chloro-benzyl amine (example 1a) using a similar procedure as described in example 15, step B.

MS ES+M+1=355.4.

### EXAMPLE 20

2-(6-amino-3-chloro-1-oxy-pyridin-2-yl)-N-(5-chloro-2-aminoethyl-benzyl)-acetamide

2-(6-amino-3-chloro-1-oxy-pyridin-2-yl)-N-(5-chloro-2-aminoethyl-benzyl)-acetamide is prepared from 2-(6-tert-Butoxycarbonylamino-3-chloro-pyridin-2-yl)-acetic acid (example 17, step A) and 2-tertButoxycarbonylaminoethyl-5-chloro-benzyl amine (example VII) using a similar procedure as described in example 15, step B.

MS ES+M+1=369.4.

### EXAMPLE 21a

2-[3-chloro-6-(2,2-difluoro-2-pyridin-2-yl-ethylamino)-pyridin-2-yl]-N-(3-chlorobenzyl)-acetamide

Step A: preparation of 2-[3-chloro-6-(2,2-difluoro-2-pyridin-2-yl-ethyl-tert-Butoxycarbonylamino)-pyridin-2-yl]-acetic Acid

2-[3-chloro-6-(2,2-difluoro-2-pyridin-2-yl-ethyl-tertButoxycarbonylamino)-pyridin-2-yl]-acetic acid is prepared from 2-(6-tert-Butoxycarbonylamino-3-chloro-pyridin-2-yl)-acetic acid ethyl ester (example 17, step A) using a similar procedure as described in example 1a, step B.

MS ES+M+1=428.3.

Step B: Preparation of 2-[3-chloro-6-(2,2-difluoro-2-pyridin-2-yl-ethylamino)-pyridin-2-yl]-N-(3-chlorobenzyl)-acetamide

2-[3-chloro-6-(2,2-difluoro-2-pyridin-2-yl-ethylamino)-pyridin-2-yl]-N-(3-chlorobenzyl)-acetamide was prepared from 2-[3-chloro-6-(2,2-difluoro-2-pyridin-2-yl-ethyl-tertButoxycarbonylamino)-pyridin-2-yl]-acetic acid (example 21a, step A) and 3-chloro-benzyl amine using a procedure similar to that described in example 1a step C.

MS ES+M+1=452.

### EXAMPLE 21b

2-[3-chloro-6-(2,2-difluoro-2-pyridin-2-yl-ethylamino)-1-oxy-pyridin-2-yl]-N-(3 chlorobenzyl)-acetamide

2-[3-chloro-6-(2,2-difluoro-2-pyridin-2-yl-ethylamino)-1-oxy-pyridin-2-yl]-N-(3 chlorobenzyl)-acetamide was prepared from 2-[3-chloro-6-(2,2-difluoro-2-pyridin-2-yl-ethylamino)-pyridin-2-yl]-N-(3 chlorobenzyl)-acetamide (example 21a, step B) using a procedure similar to that described in example 1b.

MS ES+M+1=468.

### EXAMPLE 22a

2-[3-chloro-6-(2,2-difluoro-2-pyridin-2-yl-ethylamino)-pyridin-2-yl]-N-(2-fluorobenzyl)-acetamide

2-[3-chloro-6-(2,2-difluoro-2-pyridin-2-yl-ethylamino)-pyridin-2-yl]-N-(2-fluorobenzyl)-acetamide was prepared from 2-[3-chloro-6-(2,2-difluoro-2-pyridin-2-yl-ethyl-tertButoxycarbonylamino)-pyridin-2-yl]-acetic acid (example 21a, step A) and 2-fluoro-benzyl amine using a procedure similar to that described in example 1a step C.

MS ES+M+1=435.

### EXAMPLE 22b

2-[3-chloro-6-(2,2-difluoro-2-pyridin-2-yl-ethylamino)-1-oxy-pyridin-2-yl]-N-(2-fluorobenzyl)-acetamide

2-[3-chloro-6-(2,2-difluoro-2-pyridin-2-yl-ethylamino)-1-oxy-pyridin-2-yl]-N-(2-fluorobenzyl)-acetamide was prepared from 2-[3-chloro-6-(2,2-difluoro-2-pyridin-2-yl-ethylamino)-pyridin-2-yl]-N-(2-fluorobenzyl)-acetamide using a procedure similar to that described in example 1b.

MS ES+M+1=451.

### EXAMPLE 23a

2-[3-chloro-6-(2,2-difluoro-2-pyridin-2-yl-ethylamino)-pyridin-2-yl]-N-(methyl-3-fluoro-pyridin-2-yl)-acetamide

2-[3-chloro-6-(2,2-difluoro-2-pyridin-2-yl-ethylamino)-pyridin-2-yl]-N-(methyl-3-fluoro-pyridin-2-yl)-acetamide was prepared from 2-[3-chloro-6-(2,2-difluoro-2-pyridin-2-yl-ethyl-tert-Butoxycarbonylamino)-pyridin-2-yl]-acetic acid (example 21a, step A) and 2-aminomethyl-3-fluoropyridine (Patent Publication WO 00/75134) following a procedure similar to that described in example 1a step C.

MS ES+M+1=436.

### EXAMPLE 23b

2-[3-chloro-6-(2,2-difluoro-2-pyridin-2-yl-ethylamino)-1-oxy-pyridin-2-yl]-N-(methyl-3-fluoro-pyridin-2-yl)-acetamide

2-[3-chloro-6-(2,2-difluoro-2-pyridin-2-yl-ethylamino)-1-oxy-pyridin-2-yl]-N-(methyl-3-fluoro-pyridin-2-yl)-acetamide was prepared from 2-[3-chloro-6-(2,2-difluoro-2-pyridin-2-yl-ethylamino)-pyridin-2-yl]-N-(methyl-3-fluoro-pyridin-2-yl)-acetamide using a procedure similar to that described in example 1b.

MS ES+M+1=452.

### EXAMPLE 24

2-[3-chloro-6-(2,2-difluoro-2-pyridin-2-yl-ethylamino)-1-oxy-pyridin-2-yl]-N-(methyl-3-chloro-pyridin-2-yl)-acetamide

2-[3-chloro-6-(2,2-difluoro-2-pyridin-2-yl-ethylamino)-pyridin-2-yl]-N-(methyl-3-chloro-pyridin-2-yl)-acetamide was prepared from 2-[3-chloro-6-(2,2-difluoro-2-pyridin-2-yl-ethyl-tert-Butoxycarbonylamino)-pyridin-2-yl]-acetic acid (example 21a, step A) and 2-aminomethyl-3-chloropyridine (Patent Publication WO 01/38323) following a procedure similar to that described in example 1a step C.

2-[3-chloro-6-(2,2-difluoro-2-pyridin-2-yl-ethylamino)-1-ox y-pyridin-2-yl]-N-(methyl-3-chloro-pyridin-2-yl)-acetamide was prepared from 2-[3-chloro-6-(2,2-difluoro-2-pyridin-2-yl-ethylamino)-pyridin-2-yl]-N-(methyl-3-chloro-pyridin-2-yl)-acetamide using a procedure similar to that described for example 1b.

MS ES+M+1=469.

### EXAMPLE 25a

2-[3-chloro-6-(2,2-difluoro-2-pyridin-2-yl-ethylamino)-pyridin-2-yl]-N-(3-[1,2,4]triazol-1-yl-pyridin-2-yl-methyl)-acetamide

2-[3-chloro-6-(2,2-difluoro-2-pyridin-2-yl-ethylamino)-pyridin-2-yl]-N-(3-[1,2,4]triazol-1-yl-pyridin-2-yl-methyl)-acetamide was prepared from 2-[3-chloro-6-(2,2-difluoro-2-pyridin-2-yl-ethyl-tertButoxycarbonylamino)-pyridin-2-yl]-acetic acid (example 21a, step A) and 2-aminomethyl-3-[1,2,4]triazolo-1-yl-pyridine (example V) using a procedure similar to that described for example 1a step C.

MS ES+M+1=485.

### EXAMPLE 25b

2-[3-chloro-6-(2,2-difluoro-2-pyridin-2-yl-ethylamino)-1-oxy-pyridin-2-yl]-N-(3-[1,2,4]triazol-1-yl-pyridin-2-yl-methyl)-acetamide

2-[3-chloro-6-(2,2-difluoro-2-pyridin-2-yl-ethylamino)-1-oxy-pyridin-2-yl]-N-(3-[1,2,4]triazol-1-yl-pyridin-2-yl-methyl)-acetamide was prepared from 2-[3-chloro-6-(2,2-difluoro-2-pyridin-2-yl-ethylamino)-pyridin-2-yl]-N-(3-[1,2,4]triazol-1-yl-pyridin-2-yl-methyl)-acetamide using a procedure similar to that described for example 1b.

MS ES+M+1=501.

### EXAMPLE 26

2-[3-chloro-6-(2,2-difluoro-2-pyridin-2-yl-ethylamino)-1-oxy-pyridin-2-yl]-N-(2-[1,2,4]triazol-benzyl)-acetamide

2-[3-chloro-6-(2,2-difluoro-2-pyridin-2-yl-ethylamino)-pyridin-2-yl]-N-(2-[1,2,4]triazol-benzyl)-acetamide was prepared from 2-[3-chloro-6-(2,2-difluoro-2-pyridin-2-yl-ethyl-tert-Butoxycarbonylamino)-pyridin-2-yl]-acetic acid (example 21a, step A) and 2-[1,2,4]triazol-1-yl-benzyl amine (example IX) following a procedure similar to that described in example 1a step C.

2-[3-chloro-6-(2,2-difluoro-2-pyridin-2-yl-ethylamino)-1-oxy-pyridin-2-yl]-N-(2-[1,2,4]triazol-benzyl)-acetamide was prepared from 2-[3-chloro-6-(2,2-difluoro-2-pyridin-2-yl-ethylamino)-pyridin-2-yl]-N-(2-[1,2,4]triazol-benzyl)-acetamide using a procedure similar to that described in example 1b.

MS ES+M+1=500.

### EXAMPLE 27

2-[3-chloro-6-(2,2-difluoro-2-pyridin-2-yl-ethylamino)-1-oxy-pyridin-2-yl]-N-(5-chloro-2-[1,2,4]triazol-1-yl-benzyl)-acetamide

2-[3-chloro-6-(2,2-difluoro-2-pyridin-2-yl-ethylamino)-pyridin-2-yl]-N-(2-[1,2,4]triazol-benzyl)-acetamide was prepared from 2-[3-chloro-6-(2,2-difluoro-2-pyridin-2-yl-ethyl-tert-Butoxycarbonyl amino)-pyridin-2-yl]-acetic acid (example 21a, step A) and 5-chloro-2-[1,2,4]triazol-1-yl-benzyl amine (example VIII) following a procedure similar to that described in example 1a step C.

2-[3-chloro-6-(2,2-difluoro-2-pyridin-2-yl-ethylamino)-1-oxy-pyridin-2-yl]-N-(5-chloro-2-[1,2,4]triazol-1-yl-benzyl)-acetamide was prepared from 2-[3-chloro-6-(2,2-difluoro-2-pyridin-2-yl-ethylamino)-pyridin-2-yl]-N-(5-chloro-2-[1,2,4]triazol-1-yl-benzyl)-acetamide following a similar procedure to that described in example 1b.

MS ES+M+1=534.

### EXAMPLE 28

2-[3-chloro-6-(2,2-difluoro-2-pyridin-2-yl-ethylamino)-1-oxy-pyridin-2-yl]-N-(2-aminomethyl-benzyl)-acetamide 2-[3-chloro-6-(2,2-difluoro-2-pyridin-2-yl-ethylamino)-pyridin-2-yl]-N-(2-aminomethyl-benzyl)-acetamide was prepared from 2-[3-chloro-6-(2,2-difluoro-2-pyridin-2-yl-ethyl-tertButoxycarbonylamino)-pyridin-2-yl]-acetic acid (example 21a, step A) and 2-tertButoxycarbonylaminomethyl-benzyl amine (example VI), using a similar procedure as described in example 2a.

2-[3-chloro-6-(2,2-difluoro-2-pyridin-2-yl-ethylamino)-1-oxy-pyridin-2-yl]-N-(2-aminomethyl-benzyl)-acetamide was prepared from 2-[3-chloro-6-(2,2-difluoro-2-pyridin-2-yl-ethylamino)-pyridin-2-yl]-N-(2-aminomethyl-benzyl)-acetamide using a similar procedure as described in example 2b.

MS ES+M+1=462.

### EXAMPLE 29

2-[3-chloro-6-(2,2-difluoro-2-pyridin-2-yl-ethylamino)-1-oxy-pyridin-2-yl]-N-(2-aminomethyl-5-chlorobenzyl)-acetamide

2-[3-chloro-6-(2,2-difluoro-2-pyridin-2-yl-ethylamino)-pyridin-2-yl]-N-(2-aminomethyl-5-chlorobenzyl)-acetamide was prepared from 2-[3-chloro-6-(2,2-difluoro-2-pyridin-2-yl-ethyl-tertButoxycarbonylamino)-pyridin-2-yl]-acetic acid (example 21a, step A) and 2-tertButoxycarbonylaminomethyl-5-chlorobenzyl amine (example 1a), using a similar procedure as described in example 2a.

2-[3-chloro-6-(2,2-difluoro-2-pyridin-2-yl-ethylamino)-1-oxy-pyridin-2-yl]-N-(2-aminomethyl-5-chlorobenzyl)-acetamide was prepared from 2-[3-chloro-6-(2,2-difluoro-2-pyridin-2-yl-ethylamino)-pyridin-2-yl]-N-(2-aminomethyl-5-chlorobenzyl)-acetamide using a similar procedure as described in example 2b.

MS ES+M+1=496.

### EXAMPLE 30

2-[3-chloro-6-(2,2-difluoro-2-phenyl-ethylamino)-1-oxy-pyridin-2-yl]-N-(2-aminomethyl-benzyl)-acetamide

Step A: preparation of 2,2-difluoro-2-phenyl-ethyl Trifluomethanesulfonate

To a stirred solution of (ca. 16 mmol) of ethyl 2,2-difluorophenylacetate (Patent Publication WO 99/11267) in 50 mL of absolute ethanol at 0° C. was added 3.5 g (93 mmol) of sodium borohydride in three portions. After 18 h, the reaction was quenched by the addition of 50 mL of water. The reaction mixture was concentrated at reduced pressure and the residue partitioned between 500 mL of ethyl acetate and sat. NaHCO₃. The layers were separated and the aqueous phase was washed with further portions ethyl acetate (3×). The combined organic extracts were dried over Na₂SO₄ and concentrated at reduced pressure to give an amber oil that was purified on SiO₂ using 1:1 EtOAc-hexane. All clean fractions were combined and concentrated at reduced pressure, giving the alcohol as a light yellow oil: ¹H NMR (CDCl₃) δ 2.15 (brt, J=7.0 Hz, 1H), 3.95 (dt, J=13.4, 7.1, 2H), 7.55-7.40 (m, 5H).

To a stirred solution of 1.08 g (6.84 mmol) of 2,2-difluoro-2-phenyl ethanol and 2.88 g (14.0 mmol) of 2,6-di-t-butyl-4-methylpyridine in 30 mL of methylene chloride at −78° C. under Ar was added 2.30 mL (13.7 mmol) of triflic anhydride dropwise. The reaction was allowed to gradually warm to room temperture overnight. The mixture was diluted with 100 mL of hexane and filtered. The filtrate was concentrated and treated again with hexane and filtered. Concentration of the filtrate gave the triflate as a pink oil: ¹H NMR (CDCl₃) δ 4.69 (t, J=12.0 Hz, 2H), 7.45-7.55 (m, 5H).

Step B: preparation of 2-[3-chloro-6-(2,2-difluoro-2-phenyl-ethyl-tert-Butoxycarbonylamino)-pyridin-2-yl]-acetic Acid

2-[3-chloro-6-(2,2-difluoro-2-phenyl-ethyl-tertButoxycarbonylamino)-pyridin-2-yl]-acetic acid is prepared from 2-(6-tert-Butoxycarbonylamino-3-chloro-pyridin-2-yl)-acetic acid ethyl ester (example 17, step A) using a similar procedure as described in example 1a, step B.

MS ES+M+1=427.2.

Step C: Preparation of 2-[3-chloro-6-(2,2-difluoro-2-phenyl-ethylamino)-1-oxy-pyridin-2-yl]-N-(2-aminomethyl-benzyl)-acetamide

2-[3-chloro-6-(2,2-difluoro-2-phenyl-ethylamino)-pyridin-2-yl]-N-(2-aminomethyl-benzyl)-acetamide was prepared from 2-[3-chloro-6-(2,2-difluoro-2-phenyl-ethyl-tertButoxycarbonylamino)-pyridin-2-yl]-acetic acid (above) and 2-tertButoxycarbonylaminomethyl-benzyl amine (example VI), using a similar procedure as described in example 2a.

2-[3-chloro-6-(2,2-difluoro-2-phenyl-ethylamino)-1-oxy-pyridin-2-yl]-N-(2-aminomethyl-benzyl)-acetamide was prepared from 2-[3-chloro-6-(2,2-difluoro-22-phenyl-ethylamino)-pyridin-2-yl]-N-(2-aminomethyl-benzyl)-acetamide using a similar procedure as described in example 2b.

MS ES+M+1=461.

### EXAMPLE 31

N-(3-chlorobenzyl)-2-{2-[(2,2-difluoro-2-pyridin-2-ylethyl)amino]pyrimidin-4-yl}acetamide

Step A: Preparation of ethyl [2-(methylthio)pyrimidin-4-yl]acetate

To −78° C. solution of 1 g (7 mmol) 4-methyl-2-(methylthio)-pyrimidine in 20 mL THF was added 9 mL (18 mmol, 2M solution in THF/heptane/ethylbenzene) lithium diisopropyl amide. The reaction mixture was stirred 15 minutes, then 1 mL (8.2 mmol) diethylcarbonate was added via syringe. The reaction was allowed to slowly warm to 0° C. and allowed to stir 4 hours. The reaction mixture was quenched by addition of 300 mL saturated aqueous ammonium chloride solution, extracted with 300 mL EtOAc, and the EtOAc extract dried over Na₂SO₄, filtered, and concentrated. Purification by automated flash chromatography (90 g silica gel, linear gradient 0-100% EtOAc/hex over 25 min) afforded ethyl [2-(methylthio)pyrimidin-4-yl]acetate. ¹H NMR (300 MHz, CDCl₃) δ 8.47 (d, 1H, J=4.8 Hz); 6.98 (d, 1H, J=4.8 Hz); 4.20 (q, 2H, J=7.6 Hz); 3.73 (s, 2H); 2.56 (s, 3H); 1.28 (t, 3H, J=7.6 Hz).

MS ES+M+1=213.1.

Step B: Preparation of ethyl [2-(methylsulfonyl)pyrimidin-4-yl]acetate

To a solution of 1.4 g (6.6 mmol) ethyl [2-(methylthio)pyrimidin-4-yl]acetate in 30 mL CH₂Cl₂ was added 3.7 g (12.8 mmol, 60%by wt.) m-chloroperbenzoic acid and the reaction mixture was allowed to stir 16 hours at room temperature before filtering through celite (20 mL CH₂Cl₂ rinse). The filtrate was diluted with 200 mL CH₂Cl₂ and washed 2×100 mL saturated aqueous sodium bicarbonate solution, 100 mL brine, dried over Na₂SO₄, filtered and concentrated. The residue was redissolved in 200 mL CH₂Cl₂ and washed 2×100 mL saturated aqueous sodium bicarbonate solution, 100 mL brine, dried over Na₂SO₄, filtered and concentrated to give ethyl [2-(methylsulfonyl)pyrimidin-4-yl]acetate. ¹H NMR (400 MHz, CDCl₃) δ 8.8 (d, 1H, J=5.12 Hz); 7.64 (d, 1H, J=5.12 Hz); 4.22 (q, 2H, J=7.14 Hz); 3.98 (s, 2H); 3.37 (s, 3H); 1.28 (t, 3H, J=7.15 Hz).

MS ES+M+1=245.1

Step C: Preparation of ethyl {2-[(2,2-difluoro-2-pyridin-2-ylethyl)amino]pyrimidin-4-yl}acetate

A neat mixture of 0.8 g (3.32 mmol) ethyl [2-(methylsulfonyl)pyrimidin-4-yl]acetate and 0.35 g (2.2 mmol) 2-(2-pyridyl)-2,2-difluoroethylamine was heated to 70° C. for 40 hours, cooled, diluted with 5 mL CH₂Cl₂ and purified by flash chromatography (4×14 cm silica gel, linear gradient 2-4% MeOH/CH₂Cl₂) and the mixed fractions repurified (3×15 cm silica gel, linear gradient 1-5% MeOH/CH₂Cl₂) to give ethyl {2-[(2,2-difluoro-2-pyridin-2-ylethyl)amino]pyrimidin-4-yl} acetate.

¹H NMR (400 MHz, CDCl₃) δ 8.66 (d, 1H, J=4.76 Hz); 8.22 (d, 1H, J=4.95 Hz); 7.80 (dt, 1H, J=1.84 and 7.88); 7.68 (d, 1H, J=7.88 Hz); 7.38 (dd, 1H, J=4.94 and 6.78 Hz); 6.57 (d, 1H, J=4.95 Hz); 4.41 (dt, 2H, J=6.59 and 13.91 Hz); 4.18 (q, 2H, J=7.2 Hz); 3.58 (s, 2H); 1.26 (t, 3H, J=7.2 Hz).

MS ES+M+1=323.1.

Step D: Preparation of N-(3-chlorobenzyl)-2-{2-[(2,2-difluoro-2-pyridin-2-ylethyl)amino]pyrimidin-4-yl}acetamide

To a solution of 0.07 g (0.22 mmol) ethyl {2-[(2,2-difluoro-2-pyridin-2-ylethyl)amino]pyrimidin-4-yl}acetate in 1 mL methanol was added 0.22 mL (0.22 mmol, 1M solution) aqueous LiOH. After 3 hours another 0.022 mL more LiOH solution was added and the reaction mixture was allowed to stir 16 hours, then neutralized with 0.03 mL concentrated HCl and concentrated keeping the bath <20° C. The residue was immediately dissolved in 1 mL DMF and to this was added 0.025 mL (0.2 mmol) m-chlorobenzylamine, 0.028 g (0.2 mmol) HOAt and 0.06 g (0.31 mmol) EDC. After 1 hour another 0.025 mL m-chlorobenzylamine and 0.06 g EDC were added and the reaction mixture allowed to stir 16 hours, then diluted with 30 mL EtOAc, washed with 30 mL saturated aqueous sodium bicarbonate solution, 30 mLdilute brine, 30 mL brine, dried over Na₂SO₄, filtered and concentrated. Purification by flash chromatography (2×12 cm silica gel, linear gradient 0.5-5% MeOH with 10% conc NH₄OH CH₂Cl₂) afforded N-(3-chlorobenzyl)-2-{2-[(2,2-difluoro-2-pyridin-2-ylethyl)amino]pyrimidin-4-yl}acetamide. ¹H NMR (400 MHz, CDCl₃) δ 8.58 (d, 1H, J=4.39 Hz); 8.22 (d, 1H, J=4.95 Hz); 7.79 (dt, 1H, J=1.46 and 7.69); 7.59 (br d, 1H, J=6.96 Hz); 7.38 (dd, 1H, J=4.94 and 6.96 Hz);7.20 (m, 3H); 7.15 (m, 1H); 6.56 (d, 1H, J=4.95 Hz); 5.63 (br m, 1H); 4.43 (d, 2H, J=6.04 Hz); 4.36 (dt, 2H, J=6.59 and 14.1 Hz); 3.59 (s, 2H).

### EXAMPLE 32

N-[5-chloro-2-(1H-tetraazol-1-yl)benzyl]-2-{2-[(2,2-difluoro-2-pyridin-2-ylethyl)amino]pyrimidin-4-yl}acetamide

The title compound is prepared using a similar procedure as described in example 31, step D above except 1-[5-chloro-2-(1H-tetrazol-1-yl)phenyl]methanamine (example IV) is substituted for m-chlorobenzylamine: ¹H NMR (DMSO-d₆, 400 MHz as bis-trifluoroacetate salt): δ 9.84 (s, 1H), 8.67 (d, 1H, J=4.3 Hz), 8.62 (br s, 1H), 8.20 (d, 1H, J=5.0 Hz), 7.95 (t, 1H, J=7.3 Hz), 7.67 (d, 1H, J=7.8 Hz), 7.62 (s, 2H), 7.57-7.52 (m, 2H), 7.46 (br s, 1H), 6.58 (d, 1H, J=5.0 Hz), 4.26 (dt, 2H, J=6.1 Hz and 15.1 Hz), 4.14 (d, 1H, J=5.7 Hz), 3.44 (s, 2H); HRMS (Electrospray): Calc'd for [C₂₁H₁₈ClF₂N₉O]H⁺=486.1364, Found: 486.1358; TLC: R_{f}=0.28 (160/10/1 of methylene chloride/methanol/concentrated ammonium hydroxide).

### EXAMPLE 33

N-(3-chlorobenzyl)-2-(5-cyano-2-{[2,2-difluoro-2-(1-oxidopyridin-2-yl)ethyl]amino}pyrimidin-4-yl)acetamide.

Step A: Preparation of 4-hydroxy-2-(methylthio)pyrimidine-5-carbonitrile

To a 0° C. solution of 4.5528 g potassium hydroxide in 30 mL of MeOH was added a slurry of 13.5992 g (48.9 mmol) of 2-methyl-2-thiopseudourea sulfate in 80 mL of MeOH. After 1 h at room temperature, the reaction mixture was filtered. To the filtrate, maintained at 9° C., was added 16.5267 g (97.7 mmol) ethyl(ethoxy-methylene)-cyanoacetate. After 20 min at 0° C., the mixture was filtered and the isolated solid washed with 200 mL of cold MeOH and 100 mL of cold Et₂O. This solid was treated with 100 mL of 0.5M NaOH, heated to 50° C. for 20 min, and then filtered. The filtrate was cooled to 0° C. and acidified to pH 1 with 1N HCl. The precipitate was isolated by vacuum filtration and placed under vacuum alongside P₂O₅ to give of 4-hydroxy-2-(methylthio)pyrimidine-5-carbonitrile as a yellow solid. ¹H NMR (CD₃OD, 400 MHz) δ 8.375 (s, 1H, ArH); 2.623 (s, 3H, CH₃);

MS ES+M+1=167.8.

Step B: Preparation of 4-chloro-2-(methylthio)pyrimidine-5-carbonitrile

A mixture of 7.5660 g (45.25 mmol) 4-hydroxy-2-(methylthio)pyrimidine-5-carbonitrile and 40 mL (429.14 mmol) of phosphorus oxychloride was heated at 110° C. for 3.5 h and then concentrated in vacuo. To the resulting residue was added and decanted boiling hexane (60 mL×4). The combined hexane fractions were concentrated in vacuo to give of 4-chloro-2-(methylthio)pyrimidine-5-carbonitrile as a yellow solid. ¹H NMR (CDCl₃, 400 MHz) δ 8.645 (s, 1H, ArH); 2.625 (s, 3H, CH₃);

MS ES+M+1=186.0

Step C: Preparation of ethyl [5-cyano-2-(methylthio)pyrimidin-4-yl]acetate

To a −78° C. solution of 24.4051 g (119.46 mmol) ethyl trimethylsilyl malonate in 100 mL of THF was added 47.78 mL (119.46 mmol) of 2.5M nBuLi in hexane dropwise over 20 min. After 45 min at −78° C., a soln of 7.3920 g (39.82 mmol) 4-chloro-2-(methylthio)pyrimidine-5-carbonitrile in 100 mL of THF was added dropwise over 40 min. After 20 min at −78° C., the reaction was warmed to room temperature. After 3.5 h at room temperature, a −78° C. mixture of 12.2026 g (59.73 mmol) ethyl trimethylsilyl malonate, 23.89 mL (59.73 mmol) of 2.5M nBuLi and 30 mL of THF was added dropwise over 10 min. Reaction stirred at room temperature for another 1.5 h and then quenched with 200 mL of H₂O and extracted with EtOAc (200 mL×2). The combined EtOAc fractions were washed w/ 200 mL of brine, dried over Na₂SO₄, filtered and concentrated in vacuo. The crude material was split in half and purified in two runs on the ISCO CombiFlash (90 g silica cartridge, 60 mL/min flow rate) with 0-30% EtOAc/hexane to give ethyl [5-cyano-2-(methylthio)pyrimidin-4-yl]acetate as a yellow oil. ¹H NMR (CDCl₃, 400 MHz) δ 8.692 (s, 1H, ArH); 4.239 (dd, 2H, J=7.15 Hz, 14.28 Hz, CH₂); 3.961 (s, 2H, CH₂); 2.593 (s, 3H, CH₃); 1.298 (t, 3H, J=7.15 Hz, CH₃); MS ES+M+1=238.2.

Step D: Preparation of ethyl [5-cyano-2-(methylsulfinyl)pyrimidin-4-yl]acetate

To a solution of 0.3263 g (1.38 mmol) ethyl [5-cyano-2-(methylthio)pyrimidin-4-yl]acetate in 3 mL of CHCl₃ was added 0.0859 g (0.348 mmol) m-chloroperoxybenzoic acid (70% by wt.). Within an hour, an additional 0.1647 g (0.669 mmol) M-CPBA was added in two portions. After 2 h of stirring at room temperature, the reaction was diluted with 60 mL of CH₂Cl₂, washed with 30 mL of a saturated NaHCO₃ solution and 30 mL of brine, dried over Na₂SO₄, filtered and concentrated in vacuo to give a 4.8 to 1 mixture of ethyl [5-cyano-2-(methylsulfinyl)pyrimidin-4-yl]acetate and ethyl [5-cyano-2-(methylthio)pyrimidin-4-yl]acetate. ¹H NMR (CDCl₃, 400 MHz) δ 9.099 (s, 1H, ArH); 4.333-4.186 (m, 4H, CH₂); 3.012 (s, 3H, CH₃); 1.332-1.254 (m, 3H, CH₃).

Step E: Preparation of ethyl (5-cyano-2-{[2,2-difluoro-2-(1-oxidopyridin-2-yl)ethyl]amino}pyrimidin-4-yl)acetate

To a solution of 0.0744 g (0.294 mmol) of ethyl [5-cyano-2-(methylsulfinyl)pyrimidin-4-yl]acetate in 2 mL of CH₂Cl₂ was added 0.0614 g (0.353 mmol) of 2,2-difluoro-2-(1-oxidopyridin-2-yl)ethanamine (Patent Publication WO 01/38323). After 45 min at room temperature, the reaction was diluted with 30 mL of CH₂Cl₂, washed w/ 15 mL of a saturated NaHCO₃ solution and 15 mL of brine, dried over Na₂SO₄, filtered and concentrated in vacuo. Purification by flash chromatography (20×80 mm silica gel, linear gradient 5-7% (10% NH₄OH:MeOH):CH₂Cl₂) afforded ethyl (5-cyano-2-{[2,2-difluoro-2-(1-oxidopyridin-2-yl)ethyl]amino}pyrimidin-4-yl)acetate. ¹H NMR (CDCl₃, 400 MHz) δ 8.442 (d, J=6.72 Hz, 1H, ArH); 8.288 (d, J=4.88 Hz, 1H, ArH); 7.648 (d, J=4.88 Hz, 1H, ArH); 7.397-7.302 (m, 2H, ArH); 4.780-4.645 (m, 2H, CH₂); 4.248-4.177 (m, 2H, CH₂); 3.796 (d, J=7.33, 2H, CH₂); 1.327-1.257 (m, 3H, CH₃).

Step F: Preparation of N-(3-chlorobenzyl)-2-(5-cyano-2-1 [2,2-difluoro-2-(1-oxidopyridin-2-yl)ethyl]amino}pyrimidin-4-yl)acetamide

To a solution of 0.0650 g (0.179 mmol) ethyl (5-cyano-2-{[2,2-difluoro-2-(1-oxidopyridin-2-yl)ethyl]amino}pyrimidin-4-yl)acetate in 800 uL of 1:1 THF:MeOH was added 179 uL (0.179 mmol) of a 1M solution of lithium hydroxide monohydrate in H₂O. After 3 h at room temperature, another 45 μL (0.045 mmol) of the lithium hydroxide solution was added. After an additional 18 h at room temperature, the reaction was concentrated in vacuo to give a yellow solid. Of this solid, 0.0099 g (0.029 mmol) was combined with 0.0658 g (0.037 mmol) 3-chlorobenzylamine hydrochloride salt, 0.0047 g (0.035 mmol) 1-hydroxy-7-azabenzotriazole, 0.0070 g (0.037 mmol) 1-ethyl-3-(3-dimethylaminopropyl)carbodiimide and 400 uL of DMF. After 21 h at room temperature, the reaction was purified on Gilson Preparatory HPLC to give N-(3-chlorobenzyl)-2-(5-cyano-2-{[2,2-difluoro-2-(1-oxidopyridin-2-yl)ethyl]amino}-pyrimidin-4-yl)acetamide. ¹H NMR for the TFA salt (DMSO, 400 MHz) δ 8.563 (s, 2H, ArH, NH); 8.391 (s, 1H, ArH); 8.362 (d, 1H, J=6.67 Hz, ArH); 7.578 (s, 1H, ArH); 7.523 (s, 1H, ArH); 7.384-7.235 (m, 4H, ArH); 4.538 (s, 1H, ArH); 4.313 (s, 1H, ArH); 3.640 (s, 1H, ArH); MS ES+M+1=459.1

Typical tablet cores suitable for administration of thrombin inhibitors are comprised of, but not limited to, the following amounts of standard ingredients:

| | General Range | Preferred Range | Most Preferred |
|---|---|---|---|
| Excipient | (%) | (%) | Range (%) |
| mannitol | 10-90 | 25-75 | 30-60 |
| microcrystalline | 10-90 | 25-75 | 30-60 |
| cellulose | | | |
| magnesium stearate | 0.1-5.0 | 0.1-2.5 | 0.5-1.5 |

Mannitol, microcrystalline cellulose and magnesium stearate may be substituted with alternative pharmaceutically acceptable excipients.

The thrombin inhibitors can also be co-administered with suitable anti-platelet agents, including, but not limited to, fibrinogen receptor antagonists (e.g. to treat or prevent unstable angina or to prevent reocclusion after angioplasty and restenosis), anticoagulants such as aspirin, thrombolytic agents such as plasminogen activators or streptokinase to achieve synergistic effects in the treatment of various vascular pathologies, or lipid lowering agents including antihypercholesterolemics (e.g. HMG CoA reductase inhibitors such as lovastatin, HMG CoA synthase inhibitors, etc.) to treat or prevent atherosclerosis. For example, patients suffering from coronary artery disease, and patients subjected to angioplasty procedures, would benefit from coadministration of fibrinogen receptor antagonists and thrombin inhibitors. Also, thrombin inhibitors enhance the efficiency of tissue plasminogen activator-mediated thrombolytic reperfusion. Thrombin inhibitors may be administered first following thrombus formation, and tissue plasminogen activator or other plasminogen activator is administered thereafter.

Typical doses of thrombin inhibitors of the invention in combination with other suitable anti-platelet agents, anticoagulation agents, or thrombolytic agents may be the same as those doses of thrombin inhibitors administered without coadministration of additional anti-platelet agents, anticoagulation agents, or thrombolytic agents, or may be substantially less that those doses of thrombin inhibitors administered without coadministration of additional anti-platelet agents, anticoagulation agents, or thrombolytic agents, depending on a patient's therapeutic needs.

In vitro Assay for Determining Proteinase Inhibition

Assays of human α-thrombin and human trypsin were performed by the methods substantially as described in Thrombosis Research, Issue No. 70, page 173 (1993) by S. D. Lewis et al.

The assays were carried out at 25° C. in 0.05 M TRIS buffer pH 7.4, 0.15 M NaCl, 0.1% PEG. Trypsin assays also contained 1 mM CaCl₂. In assays wherein rates of hydrolysis of a p-nitroanilide (pna) substrate were determined, a Thermomax 96-well plate reader was used was used to measure (at 405 nm) the time dependent appearance of p-nitroaniline. sar-PR-pna was used to assay human α-thrombin (Kₘ=125 μM) and bovine trypsin (Kₘ=125 μM). p-Nitroanilide substrate concentration was determined from measurements of absorbance at 342 nm using an extinction coefficient of 8270 cm⁻¹M⁻¹.

In certain studies with potent inhibitors (Kᵢ<10 nM) where the degree of inhibition of thrombin was high, a more sensitive activity assay was employed. In this assay the rate of thrombin catalyzed hydrolysis of the fluorogenic substrate benzyloxycarbonyl-Gly-Pro-Arg-7-amino-4-trifluoromethylcoumarin (Z-GPR-afc, Lewis S. D. et al. (1998) J. Biol. Chem. 273, pp. 4843-4854) (Km=²⁷ μM) was determined from the increase in fluorescence at 500 nm (excitation at 400 nm) associated with production of 7-amino-4-trifluoromethyl coumarin. Concentrations of stock solutions of Z-GPR-afc were determined from measurements of absorbance at 380 nm of the 7-amino-4-trifluoromethyl coumarin produced upon complete hydrolysis of an aliquot of the stock solution by thrombin.

Activity assays were performed by diluting a stock solution of substrate at least tenfold to a final concentration ≦0.1 Kₘ into a solution containing enzyme or enzyme equilibrated with inhibitor. Times required to achieve equilibration between enzyme and inhibitor were determined in control experiments. Initial velocities of product formation in the absence (Vₒ) or presence of inhibitor (Vᵢ) were measured. Assuming competitive inhibition, and that unity is negligible compared Kₘ/[S], [I]/e, and [I]/e (where [S], [I], and e respectively represent the total concentrations, of substrate, inhibitor and enzyme), the equilibrium constant (Kᵢ) for dissociation of the inhibitor from the enzyme can be obtained from the dependence of Vᵢ/Vᵢ on [I] shown in the following equation.

Vₒ/Vᵢ=1+[I]/Kᵢ

The activities shown by this assay indicate that the compounds of the invention are therapeutically useful for treating various conditions in patients suffering from unstable angina, refractory angina, myocardial infarction, transient ischemic attacks, atrial fibrillation, thrombotic stroke, embolic stroke, deep vein thrombosis, disseminated intravascular coagulation, and reocclusion or restenosis of recanalized vessels.

### EXAMPLE 34

Tablet Preparation

Tablets containing 25.0, 50.0, and 100.0 mg., respectively, of the following active compounds are prepared as illustrated below (compositions A-C). Active I is compound 2-[3-cyano-6-(2,2-difluoro-2-pyridin-2-yl-ethylamino)-pyridin-2-yl]-N-(2,5-dichloro-benzyl)-acetamide

| | Amount-(mg) | | |
|---|---|---|---|
| Component | A | B | C |
| Active I | 25 | 50 | 100 |
| Microcrystalline cellulose | 37.25 | 100 | 200 |
| Modified food corn starch | 37.25 | 4.25 | 8.5 |
| Magnesium stearate | 0.5 | 0.75 | 1.5 |

All of the active compound, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of active ingredient per tablet.

### EXAMPLE 35

Tablet Preparation

Exemplary compositions of compound 2-[3-cyano-6-(2,2-difluoro-2-pyridin-2-yl-ethylamino)-pyridin-2-yl]-N-(2,5-dichloro-benzyl)-acetamide (Active I) tablets are shown below:

| Component | 0.25 mg | 2 mg | 10 mg | 50 mg |
|---|---|---|---|---|
| Active I | 0.500% | 1.000% | 5.000% | 14.29% |
| mannitol | 49.50% | 49.25% | 47.25% | 42.61% |
| microcrystalline cellulose | 49.50% | 49.25% | 47.25% | 42.61% |
| magnesium stearate | 0.500% | 0.500% | 0.500% | 0.500% |

2, 10 and 50 mg tablets were film-coated with an aqueous dispersion of hydroxypropyl cellulose, hydroxypropyl methylcellulose and titanium dioxide, providing a nominal weight gain of 2.4%.

Tablet Preparation via Direct Compression

Active I, mannitol and microcrystalline cellulose were sieved through mesh screens of specified size (generally 250 to 750 μm) and combined in a suitable blender. The mixture was subsequently blended (typically 15 to 30 min) until the drug was uniformly distributed in the resulting dry powder blend. Magnesium stearate was screened and added to the blender, after which a precompression tablet blend was achieved upon additional mixing (typically 2 to 10 min). The precompression tablet blend was then compacted under an applied force; typically ranging from 0.5 to 2.5 metric tons, sufficient to yield tablets of suitable physical strength with acceptable disintegration times (specifications will vary with the size and potency of the compressed tablet). In the case of the 2, 10 and 50 mg potencies, the tablets were dedusted and film-coated with an aqueous dispersion of water-soluble polymers and pigment.

Tablet Preparation via Dry Granulation

Alternatively, a dry powder blend is compacted under modest forces and remilled to afford granules of specified particle size. The granules are then mixed with magnesium stearate and tabletted as stated above.

### EXAMPLE 36

Intravenous Formulations

Intravenous formulations of compound 2-[3-cyano-6-(2,2-difluoro-2-pyridin-2-yl-ethylamino)-pyridin-2-yl]-N-(2,5-dichloro-benzyl)-acetamide (Active I) were prepared according to general intravenous formulation procedures.

| Component | Estimated range | |
|---|---|---|
| Active I | 0.12-0.50 | mg |
| D-glucuronic acid* | 0.5-5 | mg |
| Mannitol NF | 50-53 | mg |
| 1 N Sodium Hydroxide | q.s. pH 3.9-4.1 | |
| Water for injection | q.s. 1.0 | mL |

Various other buffer acids, such as L-lactic acid, acetic acid, citric acid or any pharmaceutically acceptable acid/conjugate base with reasonable buffering capacity in the pH range acceptable for intravenous administration may be substituted for glucuronic acid.

What is claimed is:

1. A compound of the formula 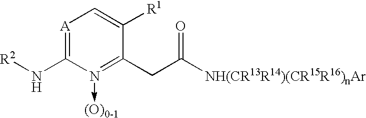
or a pharmaceutically acceptable salt thereof, wherein
R¹ is selected from the group consisting of hydrogen, halogen, cyano, C₁₋₄ alkyl, cyclo C₃₋₇ alkyl, and CF₃;
A is CH or N;
n is 0 or 1;
R² is hydrogen or —C(R⁷)(R⁸)C(R⁹)(R¹⁰)R¹¹, wherein
R⁷ and R⁸ are independently selected from the group consisting of hydrogen, halogen and C₁₋₄ alkyl unsubstituted or substituted with halogen, 0R¹², N(R¹²)₂, COOR¹², CON(R¹²)₂, aryl or a heterocyclic ring, wherein R¹² is independently selected from the group consisting of hydrogen and C₁₋₄ alkyl,
R⁹ and R¹⁰ are independently selected from the group consisting of hydrogen, halogen, and C₁₋₄ alkyl, and
R¹¹ is aryl, C₁₋₄ alkyl, C₃₋₇ cycloalkyl, or heterocycle;
R¹³ and R¹⁴ are independently selected from the group consisting of hydrogen and C₁₋₂ alkyl unsubstituted or substituted with OR¹⁵ or COOR¹⁵, wherein R¹⁵ is hydrogen or C₁₋₄ alkyl;
R¹⁵ and R¹⁶ are independently selected from the group consisting of hydrogen, halogen and C₁₋₄ alkyl; 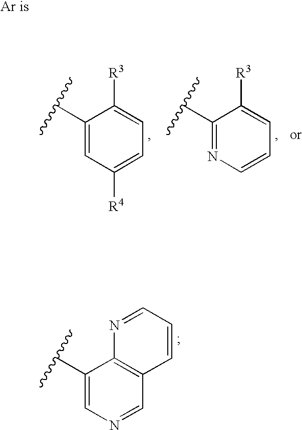
R³ is selected from the group consisting of
1) hydrogen,
2) halogen,
3) hydroxy,
4) C₁₋₄ alkyl,
5) C₁₋₄ alkoxy,
6) cyano,
7) —OCF₃,
8) —OCHF₂,
9) —OCH₂CF₃,
10) —C(R²³)(R²⁴)C(R²⁵)(R²⁶)N(R²⁷)(R²⁸) or —C(R²³)(R²⁴)N(R²⁷)(R²⁸) wherein
R²³ and R²⁴ are independently selected from the group consisting of
a) hydrogen,
b) F,
c) C₁₋₄ alkyl,
d) —CF₃,
e) —CHF₂,
f) C₃₋₇ cycloalkyl,
or R²³ and R²⁴ together form a 3-7 membered carbocyclic ring,
R²⁵ and R²⁶ are independently selected from the group consisting of
a) hydrogen,
b) C₁₋₄ alkyl
c) —CF₃,
d) —CHF₂,
e) —CH₂OH,
f) C₃₋₆ cycloalkyl,
or R²⁵ and R²⁶ together form a 3-7 membered carbocyclic ring,
R²⁷ and R²⁸ are independently selected from the group consisting of
a) hydrogen,
b) C₁₋₆ alkyl, unsubstituted or substituted with —OH, C₃₋₇ cycloalkyl, or —C(O)OR³¹, wherein R³¹ is selected from the group consisting of hydrogen and C₁₋₆ alkyl, and
c) C₃₋₇ cycloalkyl,
or R²⁷ and R²⁸ are joined to form a 4-7 membered heterocyclic ring which is unsubstituted or substituted with hydroxyl or halogen,
11) —SR¹⁷,
12) —SOR¹⁷,
13) —SO₂R¹⁷,
14) —OR¹⁸,
15) —SR¹⁸,
16) —NHR¹⁸, 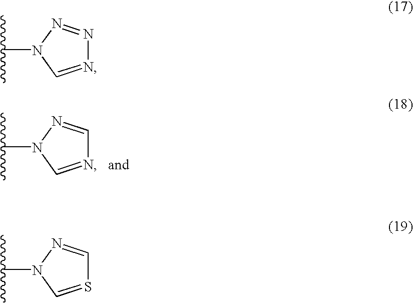
wherein
R¹⁷ is C₁₋₄ alkyl unsubstituted or substituted with —C(CH₃)₂NH₂, —C(CH₃)₂OH, —C(CH₃)₂NHCOCF₃, or CF₃, and
R¹⁸ is phenyl unsubstituted or substituted with one or more of C₁₋₄ alkyl, C₁₋₄ alkoxy, halogen, hydroxy, COOH, CONH₂, CH₂OH or COOR¹⁹, wherein R¹⁹ is C₁₋₄ alkyl;
R⁴ is selected from the group consisting of
1) hydrogen,
2) halogen,
3) hydroxy,
4) C₁₋₄ alkyl,
5) C₁₋₄ alkoxy,
6) cyano,
7) —OCF₃,
8) —OCHF₂, and
9) —OCH₂CF₃.

2. A compound of claim 1, or a pharmaceutically acceptable salt thereof, wherein R¹ is hydrogen, halogen or cyano, and n is 0.

3. A compound of claim 2, or a pharmaceutically acceptable salt thereof, wherein R⁷, R⁸, R⁹, and R¹⁰ are independently selected from the group consisting of hydrogen and halogen, and
R¹¹ is

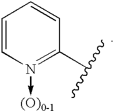

4. A compound of claim 3, or a pharmaceutically acceptable salt thereof, wherein R¹³ and R¹⁴ are hydrogen.

5. A compound of claim 4, or a pharmaceutically acceptable salt thereof, wherein R³ is selected from the group consisting of hydrogen, —CH₂NH₂, —CH₂CH₂NH₂, —CH₂NHCH₃, halogen,

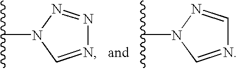

6. A compound of claim 5, or a pharmaceutically acceptable salt thereof, wherein R⁴ is hydrogen or halogen.

7. A compound of claim 6, or a pharmaceutically acceptable salt thereof, wherein
R¹ is hydrogen, Cl, or cyano; and
R² is hydrogen,

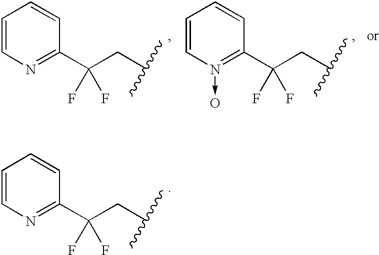

8. A compound of claim 7, or a pharmaceutically acceptable salt thereof, wherein
Ar is

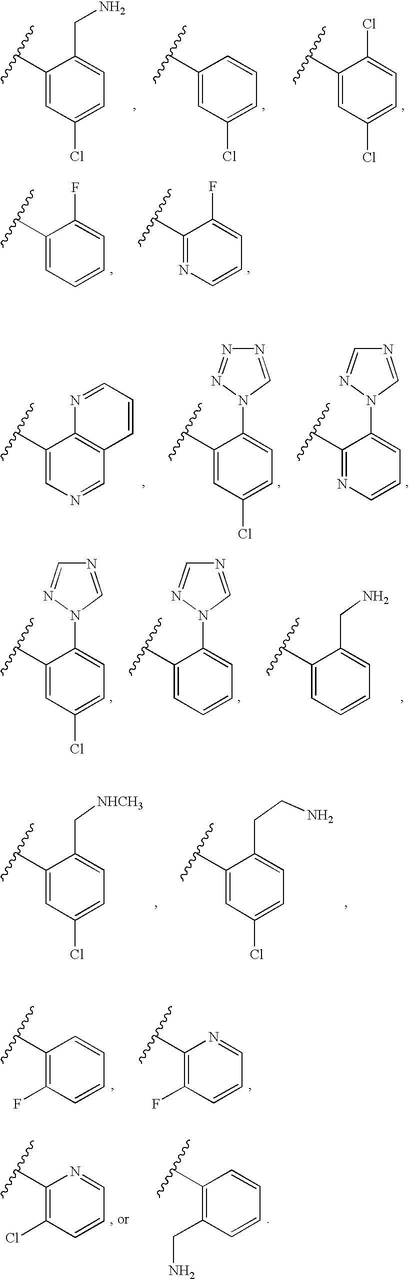

9. A compound of claim 8, or pharmaceutically acceptable salt thereof, selected from the group consisting of

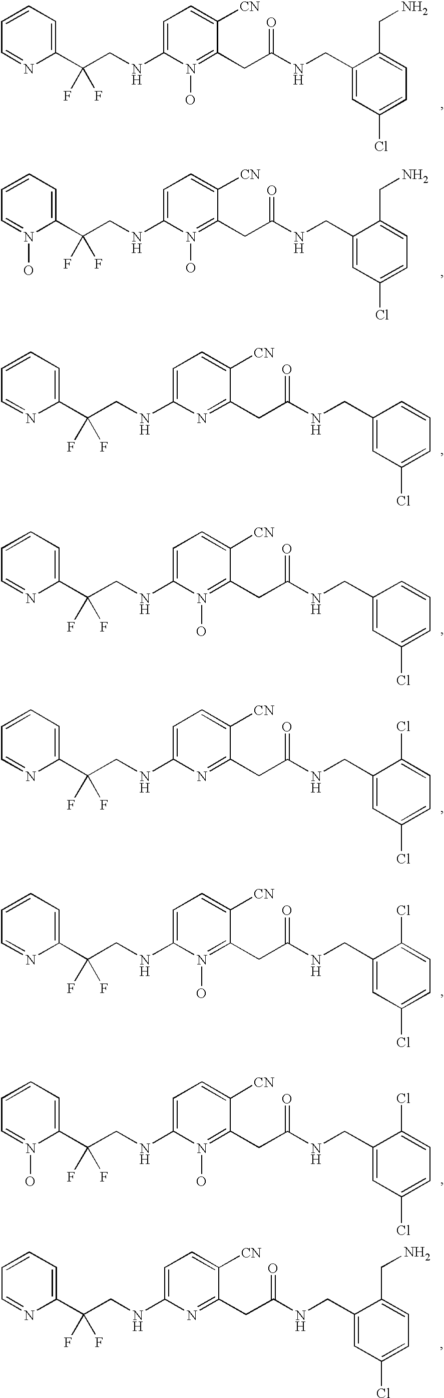

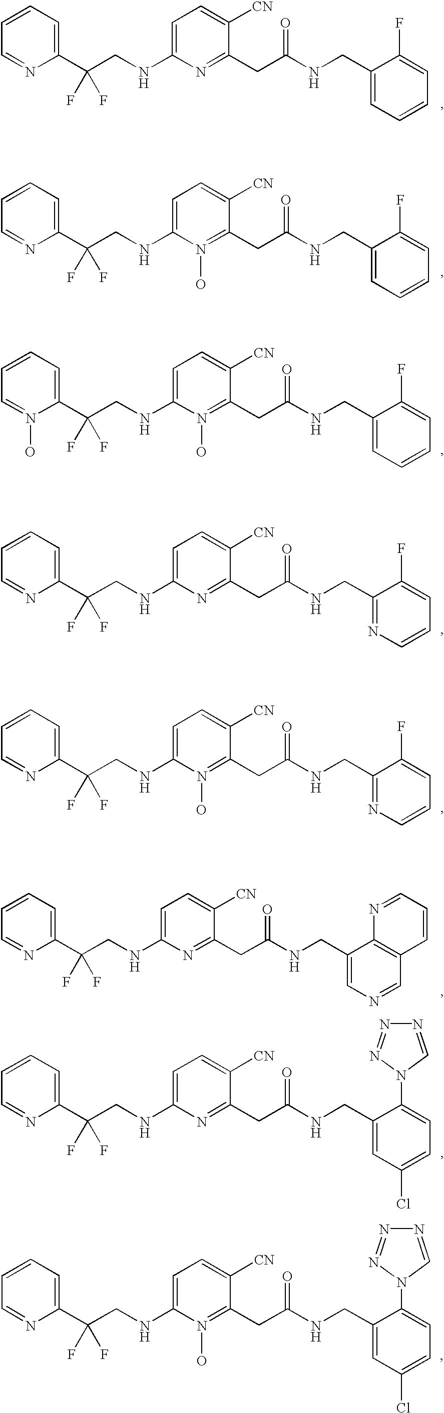

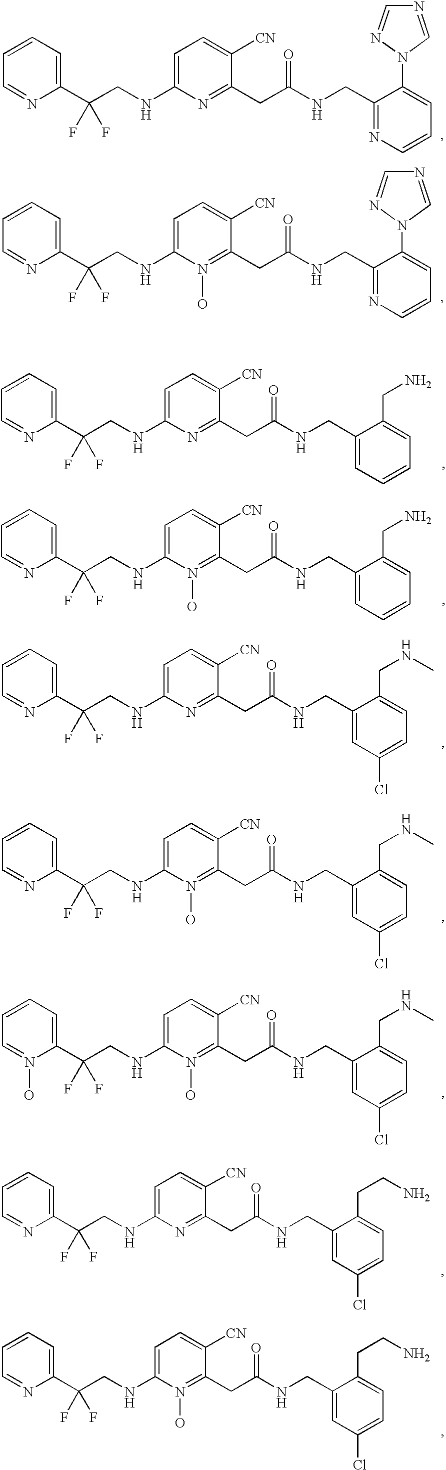

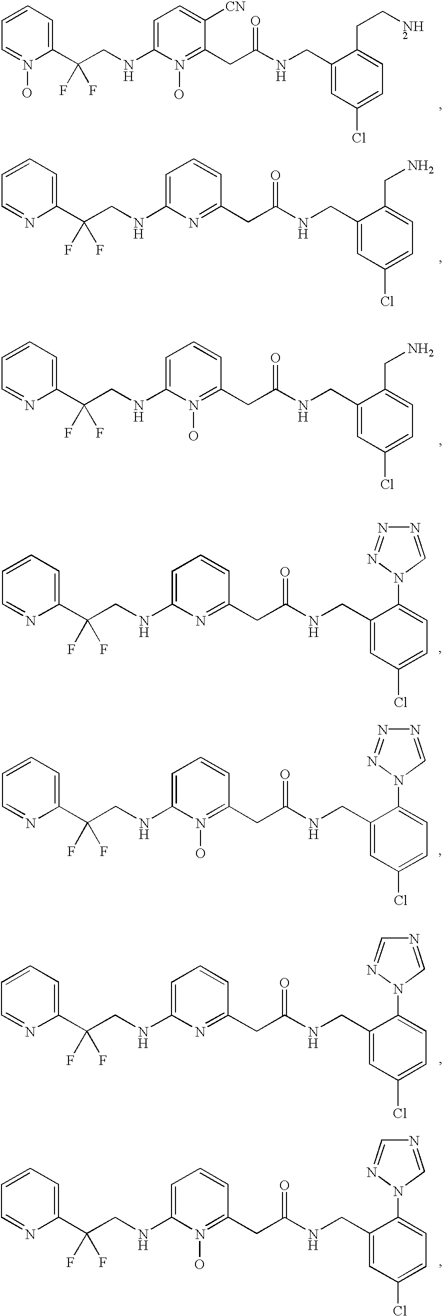

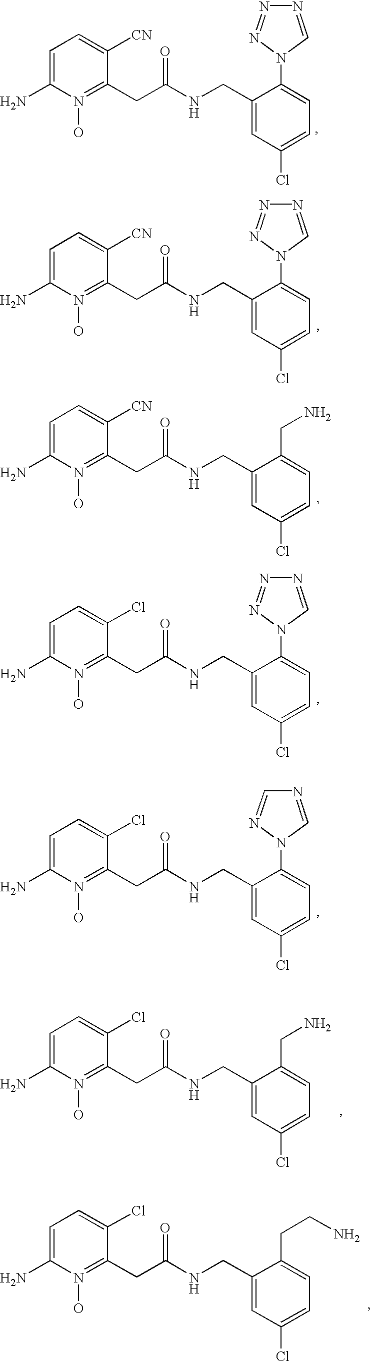

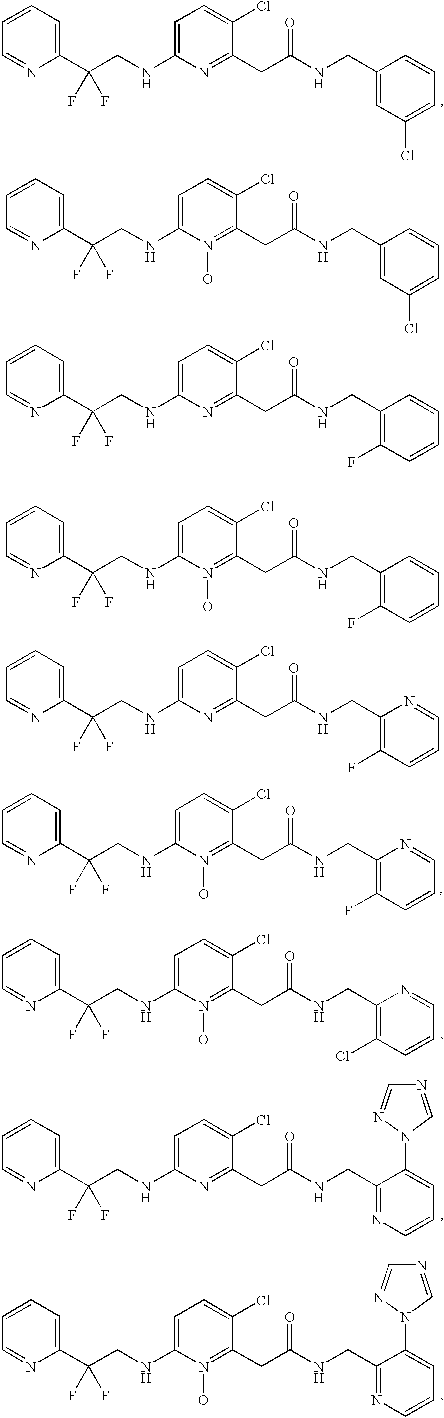

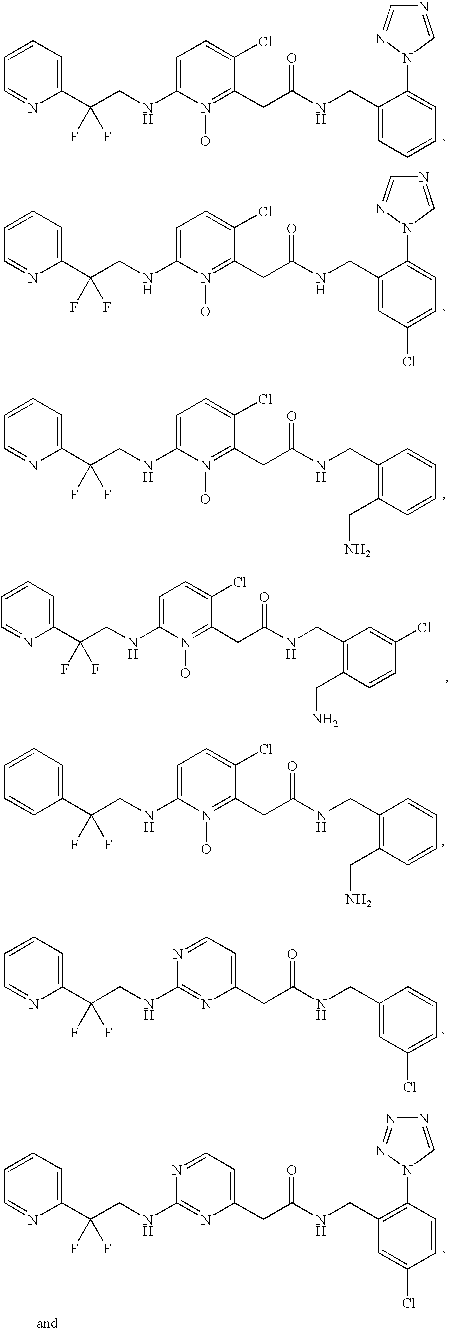

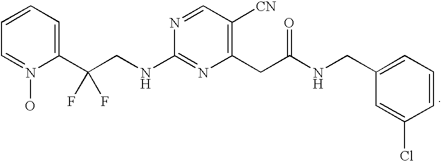

10. A composition for inhibiting thrombus formation in blood comprising a compound of claim 1 and a pharmaceutically acceptable carrier.

11. A method for inhibiting thrombin in blood comprising adding to the blood a composition of claim 10.

12. A method for inhibiting formation of blood platelet aggregates in blood comprising adding to the blood a composition of claim 10.

13. A method for inhibiting thrombus formation in blood comprising adding to the blood a composition of claim 10.

14. The use of a compound of claim 1, or a pharmaceutically acceptable salt thereof, in the manufacture of a medicament for inhibiting thrombin, inhibiting thrombus formation, treating thrombus formation, or preventing thrombus formation in a mammal.

15. A method for treating or preventing venous thromboembolism and pulmonary embolism in a mammal comprising administering to the mammal a composition of claim 10.

16. A method for treating or preventing deep vein thrombosis in a mammal comprising administering to the mammal a composition of claim 10.

17. A method for treating or preventing thromboembolic stroke in humans and other mammals comprising administering to the mammal a composition of claim 10.

18. A method for treating or preventing atrial fibrillation in a mammal comprising administering to the mammal a composition of claim 10.
